(19) United States
(12) Patent Application Publication
(10) Pub. No.: US 2024/0009647 A1
(43) Pub. Date: Jan. 11, 2024

(54) METHODS AND APPARATUS FOR INDUCING REACTIONS USING ELECTROSTATICS

(71) Applicant: Andrew Brian King, Thirsk North Yorkshire (GB)

(72) Inventor: Andrew Brian King, Thirsk North Yorkshire (GB)

(21) Appl. No.: 18/246,199

(22) PCT Filed: Sep. 21, 2021
(86) PCT No.: PCT/GB2021/052452

§ 371 Date: Mar. 22, 2023

(30) Foreign Application Priority Data
Sep. 22, 2020 (GB) GB2014961.3

(51) Int. Cl. B01J 19/08; B01J 19/26; A61K 9/50; A61K 31/00; A61K 36/185; B01J 13/14; B01F 33/05

(52) U.S. Cl. CPC B01J 19/087; B01J 19/26; A61K 9/5089; A61K 31/658; A61K 36/185; B01J 13/14; B01F 33/052; B01J 2219/00042; B01J 2219/00168; B01F 2215/0431; B01F 2215/045; B01F 2215/0445; B01F 2215/0422; B01F 2101/2204

(57) ABSTRACT

Method and associated apparatus (1) for bringing together one or more first bodies comprising a first substance with one or more second bodies comprising a second substance for the purpose of the first and second bodies, or the first and second substances contained therein, chemically and/or physically reacting together. the apparatus (1) comprising: a container (48) containing a dielectric medium (50) comprising one or more dielectric materials. especially one or more dielectric fluids or pseudo-fluids: first means (10N; 30, 36, 14, 10T) for forming the said one or more first bodies comprising the first substance and applying thereto an electric charge of a first polarity. and second means (20N; 30, 38, 24, 20T) for forming the said one or more second bodies comprising the second substance and applying thereto an electric charge of a second polarity. the first polarity being opposite to the second polarity: wherein the first and second forming means (10N, 30, 36, 14, 10T; 20N, 30, 38, 24, 20T) are each arranged for forming the respective said one or more charged first bodies and one or more charged second bodies such that each thereof is located in the said dielectric fluid medium (50) contained in the container (48), and such that the one or more charged first bodies and the one or more oppositely charged second bodies each have a size or width of at least about 0.01 mm or greater. especially of at least about 0.02 or 0.03 or 0.04 or 0.05 mm or greater;: whereby, once formed in the said dielectric medium (50), the oppositely polarised electric charges on the said first and second bodies causes or promotes electrostatic attraction between one or more respective pairs of the one or more first bodies and the one or more second bodies.

### TECHNICAL FIELD

This invention relates to methods and apparatus for inducing reactions, especially chemical and/or physical reactions, using principles of electrostatics. More particularly, though not exclusively, the invention relates to systems comprising method and apparatus aspects for effecting or inducing or facilitating chemical and/or physical reactions between at least two substances whose reaction or inter-reaction is desired, which involve the exploitation of electrostatic forces of attraction to bring the substances together under particular conditions. The invention is applicable to the carrying out of chemical and/or physical reactions between pluralities of a wide variety of substances, e.g. chemical or physical substances, species or reactants, in a wide range of industrial, chemical and pharmaceutical fields, such as in the synthesis or production of certain chemical compounds (e.g. certain polymers). pharmaceutical actives, drugs, food products and additives, and physical products, and in the production of certain synthetic fibres, alloys, electronic materials, explosives, nanomaterials, as well as possibly many other end-use substances or products in the chemical, pharmaceutical or other scientific or industrial fields.

Although it is not limited thereto, the invention is especially applicable to the carrying out of chemical and/or physical reactions between pluralities of substances, species, reactants or reagents in circumstances where anaerobic or aseptic conditions are especially important.

### BACKGROUND AND PRIOR ART

The art of chemical and pharmaceutical synthesis is replete with knowledge and disclosures of a very wide variety of methods, techniques, apparatuses, reaction environments and conditions that may be usable or preferred in various scenarios or example procedures for the production of many different kinds of such compounds or products. A particular class of chemical or pharmaceutical syntheses which generally require specialist techniques and equipment is that where anaerobic or aseptic conditions are especially important, such as in cases where one or more reagent species or reaction products are sensitive to decomposition by, or physically unstable to, oxygen, or alternatively where an aseptic reaction environment is imperative. Hitherto equipment for carrying out such reactions has generally focussed on utilising specialist reaction atmospheres or vacuums, but such apparatuses are generally bulky and expensive, they are often uneconomical to deploy and use (especially in large-scale synthesis operations), and they often come with practical limitations on how well they do their job.

In an unrelated field, electrostatics is the science of electric charges at rest and how bodies behave when charged with electrostatic charges of various magnitudes and polarities, which may give rise to electrostatic forces between such bodies. Put simply, electrostatic forces are attractive or repulsive forces between particles or bodies that are caused by their electric charges, and such forces are described by Coulomb's Law, which states that: “The magnitude of the electrostatic force of attraction or repulsion between two point charges is directly proportional to the product of the magnitudes of charges and inversely proportional to the square of the distance between them”.

The principles of electrostatic attraction and repulsion have hitherto been exploited to an extensive and highly useful level in the field of electrostatic spraying, which is well-known for the application of liquid or powder-based coating materials, e.g. of paints, colourants, surface protection layers, cleaners and disinfectants, to surfaces to be so treated. An electrostatic spraying method involves applying an electric charge (usually a positive charge) to particles—often atomised particles—of the material being applied and ground (or apply a negative charge to) the surface which is to receive the applied material. Using the basic electrostatic principle that opposite charges attract, the particles of coating materials are thus electrostatically attracted to the surface, resulting in an efficient coating procedure.

Whilst electrostatics techniques and apparatus have become widely used in a variety of industrial procedures for the manufacture and surface-treatment of materials and coated products, they have not found much use in preparative chemistry, and at most only a few studies have been published to date showing the use of electrostatic principles in the spraying of charged particles into liquids.

In one such study, as disclosed in JP2017101016A and the journal article “A Reactor System using Electrospray in the Liquid Phase and its Application in Selective Cyclosiloxane Synthesis”, Ind. Eng. Chem. Res., 2017, 56, 4878-4882, the production of siloxane oligomers from the hydrolysis of a silicon halide compound is described, in which two opposed electrospray nozzles are submerged in a low dielectric constant liquid medium and used to electrostatically spray oppositely charged ultra-fine droplets of a first liquid containing the silicon halide compound and a second liquid comprising water into the liquid medium in an electric field. As a result of collisions and fusion of the respective micro-droplets, a siloxane oligomer is formed, which rapidly diffuses into the liquid medium and thereby suppresses the molecular weight growth. However, this use of electrostatic comminution to induce siloxane oligomerisation in situ in a liquid medium is of limited interest and practical use in broader applications, because the reaction products described remain fluid and homogeneous in the liquid medium and the ultra-fine droplet sizes (typically around 8 μm) and ultra-low injection rates used are unsuitable for application in a wider context to synthesis reaction systems that use a wide variety of chemical species or reactants and which are implementable to synthesise products in practically useful amounts.

The present invention seeks to address some of the limitations and shortcomings of known techniques in the fields of chemical and pharmaceutical synthesis and certain physical reaction systems by for the first time exploiting principles of electrostatics in a novel and inventive manner.

### SUMMARY OF THE INVENTION

Accordingly, in a first aspect the present invention provides a method for bringing together one or more first bodies comprising a first substance with one or more second bodies comprising a second substance for the purpose of the first and second bodies, or the first and second substances contained therein, reacting together, the method comprising:
forming the said one or more first bodies with an electric charge of a first polarity applied thereto, and forming the said one or more second bodies with an electric charge of a second polarity applied thereto, the first polarity being opposite to the second polarity, whereby the oppositely polarised electric charges on the first and second bodies causes or promotes electrostatic attraction between one or more respective pairs of the one or more first bodies and the one or more second bodies,
wherein the one or more charged first bodies and the one or more oppositely charged second bodies are each formed in a dielectric medium comprising one or more dielectric materials,
and the one or more charged first bodies and the one or more oppositely charged second bodies each have a size or width of at least about 0.01 mm or greater, especially of at least about 0.02 or 0.03 or 0.04 or 0.05 mm or greater.

In a second aspect of the present invention there is provided apparatus for bringing together one or more first bodies comprising a first substance with one or more second bodies comprising a second substance for the purpose of the first and second bodies, or the first and second substances contained therein, reacting together, the apparatus comprising:
a container for containing a dielectric medium comprising one or more dielectric materials;
first means for forming the said one or more first bodies comprising the first substance and applying thereto an electric charge of a first polarity, and second means for forming the said one or more second bodies comprising the second substance and applying thereto an electric charge of a second polarity, the first polarity being opposite to the second polarity;
wherein the first and second forming means are each arranged for forming the respective said one or more charged first bodies and one or more charged second bodies such that the one or more charged first bodies and the one or more oppositely charged second bodies each have a size or width of at least about 0.01 mm or greater, especially of at least about 0.02 or 0.03 or 0.04 or 0.05 mm or greater, and such that each thereof is located in the said dielectric medium once contained in the container;
whereby, once formed in the said dielectric medium, the oppositely polarised electric charges on the said first and second bodies causes or promotes electrostatic attraction between one or more respective pairs of the one or more first bodies and the one or more second bodies.

In an alternative form, the above-defined apparatus of the second aspect may be provided in a condition in which the said container actually contains the said dielectric medium, in which case within the scope of the present invention, as a third aspect, is further provided apparatus for bringing together one or more first bodies comprising a first substance with one or more second bodies comprising a second substance for the purpose of the first and second bodies, or the first and second substances contained therein, reacting together, the apparatus comprising:
a container containing a dielectric medium comprising one or more dielectric materials;
first means for forming the said one or more first bodies comprising the first substance and applying thereto an electric charge of a first polarity, and second means for forming the said one or more second bodies comprising the second substance and applying thereto an electric charge of a second polarity, the first polarity being opposite to the second polarity;

wherein the first and second forming means are each arranged for forming the respective said one or more charged first bodies and one or more charged second bodies such that the one or more charged first bodies and the one or more oppositely charged second bodies have a size or width of at least about 0.01 mm or greater, especially of at least about 0.02 or 0.03 or 0.04 or 0.05 mm or greater, and such that each thereof is located in the said dielectric medium contained in the container;
whereby, once formed in the said dielectric medium, the oppositely polarised electric charges on the said first and second bodies causes or promotes electrostatic attraction between one or more respective pairs of the one or more first bodies and the one or more second bodies.

In a fourth aspect of the present invention there is provided a method for carrying out a reaction between one or more first bodies and one or more second bodies, or a first substance and a second substance contained respectively in the first and second bodies, the method comprising:
forming one or more first bodies comprising the first substance and applying thereto an electric charge of a first polarity, and forming one or more second bodies comprising the second substance and applying thereto an electric charge of a second polarity, the first polarity being opposite to the second polarity,
wherein the one or more charged first bodies and the one or more oppositely charged second bodies are each formed in a dielectric medium comprising one or more dielectric materials, and the one or more charged first bodies and the one or more oppositely charged second bodies each have a size or width of at least about 0.01 mm or greater, especially of at least about 0.02 or 0.03 or 0.04 or 0.05 mm or greater; and
allowing one or more respective pairs of the one or more charged first bodies and the one or more oppositely charged second bodies to move towards and come together with one another by virtue of electrostatic attraction therebetween caused or promoted by the oppositely polarised electric charges on the first and second bodies;
whereby the coming together of the one or more respective pairs of the one or more charged first bodies and the one or more oppositely charged second bodies enables the first and second bodies, or the first and second substances contained therein, to react together.

In practising some embodiments of the above methods of the first and fourth aspects, the bringing or coming together of the first and second bodies may result in a joining or merging or combining together of the first and the second bodies so as to form one or more product bodies, wherein the one or more product bodies comprise both the first and the second substances, which first and second substances are thereby able to interact and/or react together within the one or more product bodies. Thus, in such embodiments of the invention the one or more product bodies resulting from the joining or merging or combining together of the first and the second bodies may comprise a product of a chemical interaction or reaction between the first and the second substances originally present in the first and second bodies, respectively.

However, in practising some other embodiments of the above methods of the first and fourth aspects, the bringing or coming together of the first and second bodies may result in a joining or merging or combining together of the first and the second bodies so as to form one or more product bodies of a different kind, in which the one or more product bodies comprise discrete portions or components (e.g. of the nature of a shell-and-core or encapsulated droplet or particle or body structure), each of which portions or components contains a respective one of the first and second substances, but which first and second substances remain separate from each other within the one or more product bodies. Thus, in such embodiments of the invention the one or more product bodies resulting from the joining or merging or combining together of the first and the second bodies may comprise a product of a physical interaction or reaction between the first and the second bodies, rather than a chemical one between the first and second substances contained therein.

Thus, in practising embodiments of the present invention it is to be understood that the “reacting together” or the “reaction between” the one or more charged first bodies and the one or more oppositely charged second bodies may in many embodiment cases be, or may be predominantly, a chemical reaction between the first and second substances contained in the first and second bodies, whereby the electrostatically brought together first and second bodies enable the first and second substances to chemically react together to produce or form one or more reaction products, the desirability of synthesising which under the novel conditions of the invention may be the primary aim of such embodiments. However, it is to be understood that such “reacting together” or the “reaction between” the one or more charged first bodies and the one or more oppositely charged second bodies may, in some other embodiment cases, involve or comprise or be, at least to some degree, just a physical reaction between the said first and second bodies, whereby the electrostatically brought together first and second bodies (with their respective first and second substances contained therein) physically come and/or react together to form new product bodies comprising discrete portions or components (e.g. of the nature of a shell-and-core or encapsulated droplet or particle or body structure), each of which portions or components contains a respective one of the first and second substances, but which first and second substances remain separate from each other within the one or more product bodies. In this latter manner, it is to be understood that certain embodiments of the invention may be used to create reaction product(s) which are of a novel physical form, configuration or structure, rather than of a novel chemical identity.

As used herein the term “dielectric material” means a material that is or comprises a substance that has or exhibits a dielectric property, namely it being or behaving as an electrical insulator (or very poor conductor) that can be polarized by an applied electric field. By its very nature the atoms or molecules of the dielectric material have substantially no loosely bound, or free, electrons that may drift through the material. Thus, when the material is placed in an electric field, such as that created by the electric charges of opposite polarities applied to the bodies of the first and second substances in embodiments of the present invention, substantially no electric current flows in it (provided the material's breakdown voltage/potential is not exceeded).

In embodiments of the present invention the dielectric medium may be in the form of a dielectric fluid or a dielectric pseudo-fluid (i.e. a dielectric composition that comprises one or more solid substances but which behaves like a fluid).

In many such embodiments of the invention the dielectric medium may comprise a dielectric fluid, especially a dielectric fluid comprising one or more liquid dielectric materials. However, in other such embodiments of the invention the dielectric medium may comprise a dielectric pseudo-fluid, especially a dielectric composition that comprises one or more solid, especially particulate or powder, substances but which behaves like a fluid, wherein the one or more solid (especially particulate or powder) substances comprise or carry one or more liquid or solid dielectric materials.

For use in those embodiments of the present invention which employ a dielectric medium in the form of a dielectric fluid, the one or more dielectric fluids may be or comprise one or more dielectric liquids (i.e. liquid at room temperature (20° C.)). However, in certain other embodiments still within the scope of the invention at least one of the one or more dielectric fluids may be a gas or vapour at room temperature.

Suitable dielectric fluids or pseudo-fluids (especially dielectric liquids) for use in the dielectric medium in various embodiments of the invention may include any dielectric fluid (or pseudo-fluids) having a suitably high specific resistance, e.g. in the range of from about 10-8 Ωcm to about 10-15 Ωcm, but typically in excess of about 10-12 Ωcm). Suitable dielectric fluids/pseudo-fluids (especially dielectric liquids) may also have a high enough electric breakdown voltage/potential which is sufficient to allow a desired distance separation of, or gap between, first and second nozzle devices (or other body formation means) used to form or deliver the respective first and second bodies into the dielectric reaction medium (which desirable distance separations may for example be of from about 3 or 4 or 5 or 6 or 7 or 8 mm up to about 10 or 12 or 15 or 20 or 25 or 30 mm, and in many cases may generally be in the approx. range of 8-15 mm). Such desirable electric breakdown voltages/potentials may thus typically be in the range of from about 150 V/mm up to about 30 kV/mm, or even up to about 75 or 80 or 90 kV/mm, but more typically >2 kV/mm.

In an extension of the electrical parameters in the preceding paragraph, but still within the ambit of the present invention, it may be possible to use significantly higher voltages by moving nozzle delivery/charging devices of the apparatus significantly further apart. Under this approach the first and/or second bodies may carry a significantly higher specific charge, but due to the inverse square law may be too far apart to be attracted to each other by electrostatic attraction. However, by engineering flow regimes in the dielectric medium such that charged bodies flow towards each other after they have detached from their respective nozzle delivery/charging devices, in so doing they get to a point where the force of electrostatic attraction takes over, which thus causes the bodies to merge and react. This methodology may have some benefits in that, firstly, submerged arcing may be eliminated which may otherwise be a problem for sensitive materials, and secondly, even larger bodies may be able to be formed (especially of the shell-and-core type) in which the mass ratio of active core to containment shell may be significantly higher (e.g. approaching the size of conventional tablet capsules and single dose confectionary). It may also be expected that the higher the potential discharge on the bodies' merging the higher the turbulence between the reactant first and second substances contained in the bodies, which may result in their reaction happening faster.

Suitable dielectric liquids for use in embodiments of the invention may have a relatively low kinematic viscosity, e.g. in the range of from about 1 cSt to about 30 cSt, although typically <<30 cSt.

By way of some specific examples of the above electrical properties of some suitable dielectric liquids for use in certain embodiments of the invention:
for a dielectric liquid being for example Midel 7131, which is a synthetic ester of a natural oil produced by M&I Materials Ltd, its breakdown voltage for typical charging electrodes is >75 kV (to IEC 60156 specification), and kinematic viscosity 29 cSt at 40° C., and may typically be suitably used with a charging electrode/delivery nozzle gap of around 13 mm;
for a dielectric liquid being deionised water, its breakdown voltage (IEC 60156 specification) is 65-70 kV/mm, and its kinematic viscosity 1 cSt, and may typically be suitably used with a charging electrode/delivery nozzle gap of around 13 mm;
for a dielectric liquid being silicone oil, its breakdown voltage (IEC 60156 specification) is 10-15 kV/mm, and its kinematic viscosity in the range 2-5000 cSt (but preferably as low as possible), and may typically be suitably used with a charging electrode/delivery nozzle gap of around 13 mm.

In practising some of the above embodiments, depending on the solvent system used the dielectric fluid or pseudo-fluid may not absorb water, ethanol or other solvents which could otherwise have the effect of reducing the dielectric properties of the fluid and deleteriously affecting its electrical breakdown properties. Thus, if in certain embodiments of the invention such solvent absorption does occur, then the reaction system or medium may include means for reducing the presence or effects of such absorbed solvents, e.g. one or more desiccant materials (examples of which are readily available in the art).

Furthermore, suitable dielectric liquids for use in embodiments of the invention may have a low flash point, e.g. in the range of from about 50° C. up to about 500° C. or above, but typically about >125° C. (and/or not combustible at all).

In various such embodiments of the invention the one or more dielectric fluids or pseudo-fluids may usefully be non-biodegradable.

In various such embodiments of the invention the one or more dielectric fluids or pseudo-fluids may be either lower in density than water or higher in density than water (or possibly even the same density as water).

For use in various such embodiments of the invention the one or more dielectric fluids or pseudo-fluids may be either hydrophobic (i.e. oliophilic) or hydrophilic. Suitable hydrophobic (i.e. oliophilic) dielectric fluids may include various oils or liquid hydrocarbon-based or hydrocarbon-rich organic compounds. Suitable hydrophilic dielectric fluids may include, for example, deionised water.

Some more specific examples of the above classes of suitable dielectric liquids for use in such embodiments of the invention may include any of the following:
silicones or silicone oils, e.g. dimethicone;
hydrocarbons, e.g. paraffin, hexane, heptane;
halogenated (e.g. chlorinated and/or fluorinated) hydrocarbons, e.g. perchloroethylene, or any of the “Fluorinert” [trade mark] series of electrically insulating, stable fluorocarbon-based fluid coolant liquids sold by 3M (such as those fluorinated or perfluorinated organic compound-based products or product mixtures sold under the designations FC-40, FC-43, FC-70, FC-72, FC-75, FC-770, FC-3283 and FC-3284);
any of the “Novec” [trade mark] series of engineered dielectric fluids sold by 3M;
perether fluorocarbons, e.g. as used in pharmaceutical products, such as that sold under the trade name “Fomblin” (originally manufactured by Austmont but currently available through Solvay);
esterified natural or vegetable oils, such as those known and used as transformer oils (e.g. those sold by M&I Materials under the trade mark “Midel”);
reactive methyl hydrogen siloxate, such as the polymethylhydrogensiloxane-based water repellent product sold by PennWhite Silicones under the designation “PW23”;
mineral oils;
castor oil;
food-grade vegetable, nut or seed oils (e.g. almond oil);
polyphosphazines (i.e. any of various hybrid inorganic-organic polymers with an inorganic backbone consisting of alternating phosphorus and nitrogen atoms separated by alternating single and double bonds, and organic substituents covalently bonded to the phosphorus atoms as side groups);
certain medium-chain (i.e. C4 or C5 up to C10 or C12) fatty acids, e.g. caprylic acid;
certain carboxylic and other acid esters, e.g. trimethyl citrate, trimethyl lactate, trimethyl phosphate;
admixtures of certain oils with other organic solvents, such as admixtures of silicone oil and methylene chloride (e.g. which have variable density according to the silicone to methylene chloride ratio);
super-oleophobic materials being fluorinated surfactants (such as those used in greaseproofing agents in food contact papers or as coatings for cosmetic applications which repel skin oils), for example polyfluoroalkyl phosphates;
deionised water.

Dielectric fluids or pseudo-fluids suitable for use in such embodiments of the invention may be used either in their substantially pure, as-produced/sold form, or at they may have incorporated into them one or more additives (e.g. in a total amount of up to about 0.01 or 0.1 or 0.5 or 1 or 2 or 3 or 4 or 5 or 8 or 10% by weight) for the purpose of, for example, adjusting the density of the dielectric fluid(s)/pseudo-fluid(s) of the dielectric medium (e.g. so that its density, relative to the density of reaction product(s) produced by the reaction between the first and second bodies, is such that that/those reaction product(s) can be made to either float or sink in the dielectric reaction medium and thus travel away from the reaction zone, which may thereby assist in the reaction product(s)' isolation and recovery), adjusting the volume resistivity or one or more other parameters of the dielectric fluid(s)/pseudo-fluid(s) of the dielectric medium, or for acting as an additional reactant, or promotor or catalyst, for reacting with either the charged first substance bodies or the charged second substance bodies.

It may even be possible, in certain embodiments, for molecules of the dielectric fluid/pseudo-fluid itself to react with either the first or the second substances in the first or second bodies, or to otherwise play a role in, or be involved in (e.g. as a promotor or catalyst), the reaction between the first and the second substances in the first and second bodies.

In some such embodiments of the invention the dielectric medium may comprise substantially just a single dielectric fluid or pseudo-fluid. Thus, in such embodiments the charged bodies of the first substance may be formed in the same phase, layer or volume of the dielectric medium as are formed the charged bodies of the second substance.

However, in some other such embodiments the dielectric medium may comprise a plurality of different dielectric fluids or pseudo-fluids. In such embodiments comprising a plurality of dielectric fluids or pseudo-fluids, each dielectric fluid/pseudo-fluid may form, or be present in the container of the apparatus as, its own discrete phase or layer or volume which is separate or discrete from the phase, layer or volume forming the other, or at least one or more of the other, dielectric fluids/pseudo-fluids. Thus, in such embodiments the charged bodies of the first substance may be formed in a different phase, layer or volume of the dielectric medium from the phase, layer or volume in which are formed the charged bodies of the second substance. For example, where plural different dielectric fluids/pseudo-fluids are employed, they may be designed (e.g. by virtue of their differing densities or hydrophobicities) to form discrete upper and lower (or even upper, lower and one or more intermediate) layers in the overall dielectric medium. Alternatively, such plural different dielectric fluids/pseudo-fluids may perhaps be designed to form discrete phases of a suspension or emulsion (or even a colloid), but desirably only if dielectric breakdown is able to be avoided in such a system.

For use in those embodiments of the present invention which employ a dielectric medium in the form of a dielectric pseudo-fluid, the one or more dielectric pseudo-fluids may be or comprise a dielectric composition which comprises one or more solid substances, especially one or more particulate or powdered solid substances, comprising or carrying one or more dielectric materials, in particular one or more liquid or solid dielectric materials.

In some embodiment forms of such dielectric pseudo-fluids, the particulate or powdered solid substance(s) that acts like a fluid may itself be or comprise one or more solid dielectric materials. However, in other embodiment forms of such dielectric pseudo-fluids, the particulate or powdered solid substance(s) that acts like a fluid may be itself substantially inert (in the context of the reaction system in question) and may constitute a substrate which carries—especially carries by virtue of being impregnated with or having absorbed or adsorbed therein or thereon—one or more liquid dielectric materials. Examples of such liquid dielectric materials in this context may be any of those listed above for use as or as a component of a dielectric fluid.

As with the use of dielectric fluids, in certain embodiments it may even be possible for molecules of a solid (e.g. particulate or powder) or liquid dielectric material in a dielectric pseudo-fluid itself to react with either the first or the second substances in the first or second bodies, or to otherwise play a role in, or be involved in (e.g. as a promotor or catalyst), the reaction between the first and the second substances in the first and second bodies.

In some embodiments utilising dielectric pseudo-fluids, different particulate or powdered solid (or substrate) substances which act like a fluid may be used depending on the chemical nature of the first and second substances to be chemically reacted together. For example:
For first and/or second reactant substances which do not comprise or contain a surface tension-lowering substance, e.g. ethanol, a superhydrophobic powdered silica aerogel such as that used for the synthesis of “dry water” powder may be used. “Dry powdered” gel formulations incorporating hydrophilic gelling agents such as guar gum, gellan, xanthan gum, polyvinyl acetate, polyvinyl alcohol, polyvinyl pyrrolidone, alginate, or carboxyvinyl or carboxymethyl polymer may also be used, and such materials are known per se in the art.
Alternatively, a silicone oil such as methicone and dimethicone absorbed into a porous inert particulate or powdered substrate material (especially an inert ceramic or glass material, or an inert polymer or inert natural or plant-derived material), especially at a loading sufficient for free flow of the particulate/powder/may be used.
Alternatively, a silicone powder may be used, such as those described in U.S. Pat. No. 7,875,699B2, or a silicone polymer coated powder.
Where a low molecular weight solvent such as ethanol is used in the reaction system, polyalphaolefins with a kinematic viscosity (@100° C.) of greater than about 5 or 10 mm2 s⁻¹ (e.g. as sold by ExxonMobil under the trade marks Spectrasyn™ and Spectrasyn Elite™) may be used as a superhydrophobic phase of a “liquid-on-powder” type dielectric pseudo-fluid. (Polyalphaolefins are highly hydrophobic and also have low to zero solubility for low molecular weight aliphatic solvents like methanol, ethanol, propanol and butanol.) Alternatively, an ethanol-insoluble solvent may instead be used.

In the above examples of possible dielectric pseudo-fluids that are of the “liquid-on-powder” type, the solid particulate/powder substrate phase may be any free-flowable particulate or powdered material onto/into which the liquid dielectric material is absorbed or adsorbed (especially to produce a surface coating thereof) and which is inert to the reactive first and second substances to be reacted together. Suitable examples of such substrate materials may include: kieselgur, powdered glass, powdered polymers including polyolefins, PET, polycarbonates, and also naturally occurring materials including: powdered cork, wood, lignin, cellulose, or stone or ceramic materials.

Where they are used, particulate or powdered dielectric materials or their solid substrates, as the case may be, may preferably be substantially electrically non-conductive, in order to prevent or guard against short-circuiting between electrode nozzles (or other charging/delivery means) used to form and charge the bodies of the first and second reactive substances. Such particulate or powdered dielectric materials or their solid substrates may preferably also be substantially non-flammable, in order to prevent or guard against ignition during application of electrostatic voltages to the particulate/powder material(s). Alternatively for this same purpose, especially where there is any possibility of ignition occurring, in-powder electrostatic reactions may be carried out under an atmosphere of nitrogen, or other inert gas.

One particularly useful example of a dielectric pseudo-fluid is a superhydrophobic silica aerogel. For use in certain embodiments of the invention, silica aerogel powder, otherwise known as fumed silica, made by pyrolitic, plasma or supercritical means, may generally be provided with a particle size range of about 10-20 nm, but it can form agglomerates up to 100-500 nm or even up to a few microns (μm) in size. Such a particulate/powdered dielectric may in some embodiment cases have a particle size range of approx. 1 to 10 μm, although in other embodiment cases it could have a particle size range of from about 20 or 30 or 40 or 50 up to about 100 or 200 μm, or possibly even a particle size of up to around 0.5 or 1 mm or perhaps even as much as up to around 0.2 or 0.3 or 0.4 or 0.5 cm. Silica aerogels have a very high dielectric breakdown voltage equivalent to PTFE, which therefore may make them useful candidates for dielectric pseudo-fluid materials for use in certain embodiments of this invention. Once such silica aerogel dielectric pseudo-fluid materials have been employed in reaction methods of the invention to carry out the reaction between the first and second substances, the reaction product(s) may be freed of adhering aerogel powder by simple washing, e.g. using an ethanol+water mixture, which breaks the surface tension of the aerogel making it washable.

Another particularly useful example of a dielectric pseudo-fluid is a polyfluoroalkyl phosphate-coated powder, which may be used to good effect where the reactive first and second substances are oil-based or oleophilic materials.

In embodiments of the apparatus aspect of the invention, the container containing, or for containing, the dielectric medium may take any suitable physical form, size, shape and configuration. For example, the container may take the form of a reaction vessel of the nature of a covered or uncovered bath, cuboidal tank, or chamber, flask, tube, cylinder or other receptacle or reservoir.

In many practical embodiments of the invention the container which forms the reaction vessel may include separation or collection means for enabling product bodies resulting from the bringing together of, and reaction between, the first and second bodies to be collected and separated out from the dielectric medium, ready for subsequent removal or extraction from the container or reaction vessel. For example, a dedicated collection or separation portion of the container may be designed for this purpose, and/or a suitable arrangement of an outlet port, tube, pipe, channel or other conduit leading to the exterior of the container may be provided also for this purpose.

In some such embodiments in which product bodies resulting from the bringing together of, and reaction between, the first and second bodies themselves have or carry a residual electric charge, their separation or collection, and thus ultimately their removal from the system, may be facilitated if desired by the application of an opposite-polarity electric voltage or charge to the said separation or collection means, in order to promote or cause movement of the residually-charged product bodies towards the said separation or collection means, from where they may be more readily and efficiently removed or extracted.

In accordance with the invention the one or more first bodies comprising the first substance are formed along with—especially either substantially simultaneously with or just prior to (e.g. up to a few, e.g. up to about 1 or 2 or 3 or 5 or 10 or 20 or 30 or 40 or 50 or 60, seconds or maybe up to a few, e.g. up to about 1 or 2 or 3 or 5 or 10, minutes prior to) or just subsequent to (e.g. up to a few, e.g. up to about 1 or 2 or 3 or 5 or 10 or 20 or 30 or 40 or 50 or 60, seconds or maybe up to a few, e.g. up to about 1 or 2 or 3 or 5 or 10, minutes subsequent to)—the formation of the one or more second bodies comprising the second substance, with the aim of the invention being to allow or promote or facilitate the bringing together of those first and second substances for the purpose of their chemically reacting together.

The first bodies formed comprising the first substance may take any suitable physical form, such as droplets, globules, capsules, particles, chains, threads, fibres, or possibly other shapes or configurations. Likewise, the second bodies formed comprising the second substance may take any suitable physical form, again such as droplets, globules, capsules, particles, chains, threads, fibres, or possibly other shapes or configurations. The first and the second bodies may either be generally substantially of the same general physical form, shape or configuration, or they be different from one another.

Furthermore, in certain embodiments of the invention it may be possible for at least one of the first or second body species to be formed as a core body (e.g. taking any of the abovementioned forms of a droplet, globule, particle, chain, thread, fibre, etc) and at least one of the other first or second body species to be formed as a hollow or shell body which surrounds or encapsulates or enshrouds or encases the core body. In this manner, it is within the scope of certain embodiments of the invention for at least one of the first or second body species to be formed in the form of a hollow body (e.g. a sphere or cylinder), or a coating, sheath, covering, casing, or other encapsulating or enshrouding or encasing layer.

In practising various embodiments of the invention the one or more first bodies may be formed in any desired number(s), e.g. as either a single discrete first body or alternatively as a plurality of such first bodies. In the case of a plurality of such first bodies, they may be formed in any suitable number, e.g. as many as may be desired or appropriate in view of the nature of the chemical reaction that is to take place with the second bodies of the second reactive substance or the degree of interaction that is desired between the two reactive species. Likewise, in the case of a plurality of such second bodies, they may (independently of the first bodies) be formed in any suitable number, e.g. as many as may be desired or appropriate in view of the nature of the chemical reaction that is to take place with the first bodies of the first reactive substance or the degree of interaction that is desired between the two reactive species.

Thus, in practising many such embodiments of the invention, where the first charged bodies and/or the second charged bodies are each, either, or both formed in respective pluralities thereof, the resulting formed first and/or second bodies may be present or distributed within the dielectric medium in the form of dispersed droplets, capsules, microcapsules, particles or fibres, and of any suitable droplet/particle/fibre size or width.

According to the invention the first charged bodies and/or the second charged bodies each (which is to say, within each species) have a size or width (i.e. diameter or transverse width) of at least about 0.01 or greater, especially of at least about 0.02 or 0.03 or 0.04 or 0.05 mm or greater, more especially still of at least about 0.1 mm or greater.

In many practical embodiments of the invention the first charged bodies and/or the second charged bodies may each have a size or width (i.e. diameter or transverse width) in a range of from about 0.01 or 0.02 or 0.03 or 0.04 or 0.05 or 0.06 or 0.07 or 0.08 or 0.09 or 0.1 or 0.2 or 0.3 or 0.4 or 0.5 mm up to about 2 or 3 or 5 or 6 or 7 or 8 or 9 or 10 or 15 or 20 mm. For such smaller sized droplets or particles present in respective pluralities thereof, they may in some example embodiments even be present in the form of an emulsion in the continuous phase being the dielectric medium, or possibly even as colloidally dispersed such droplets/particles.

For defining and measuring such body sizes or widths (i.e. diameters or transverse widths), in particular where body size/width distributions over a range of size/width values are encountered, typically a mass median average size/width value may be used to define same, that being definable and measurable using any conventional technique and/or apparatus as known and used in the art for that purpose.

In many practical embodiments of the invention the one or more first bodies may comprise a first composition, especially a first liquid composition, comprising or including the said first substance, optionally together with one or more other, or auxiliary, components of the first composition, e.g. one or more solvents, co-solvents, diluents, emulsifiers, stabilisers, viscosity modifiers, polymers or gel-forming materials (especially cross-linkable such substances), cross-linking agents, dispersants/surfactants or surface tension adjusting agents, catalysts, preservatives, pH adjusting agents, pigments, flavours/fragrances, gas generating agents, photoinitiators, magnetic materials, controlled release aids, etc.

Likewise, in many practical embodiments of the invention, the one or more second bodies may comprise a second composition, especially a second liquid composition, comprising or including the said second substance, optionally together with one or more other, or auxiliary, components of the second composition, which auxiliary components may, in the context of the second composition, be independently selected from any of those classes or types or examples of auxiliary components that may be used in the first composition, as listed above.

Examples of some of the aforementioned auxiliary components that may be used independently as auxiliary components in either or both of the first composition and/or the second composition may, independently in each composition, include any of the following:
solvents may include: water, ethanol, methanol, isoproyl alcohol, pentanol, butanol, ketones (such as acetone), dimethylsulfoxide, acetamide, ethyl acetate, ethyl formate, formamide, terpineol, toluene, cyclohexanone, carbonate esters (such as propylene carbonate and glycerol carbonate), methylene chloride and other chloro- and fluoro-solvents, natural essential oils (including moringa, milk thistle, orange, caprylic to octanoic oils);
co-solvents may include: polyethylene and polypropylene glycols, higher glycols and glycol derivatives (including diglycidyl ethers of ethylene glycol and polyethylene glycol);
cross-linkable organic or inorganic polymers or gel-forming materials may include: chitosan and its derivatives (including carboxymethyl caproic and caprylic chitosan), soluble lignin and its derivatives, alginate, hyalauronic acid, carrageenan, locust bean gum, xanthan gum, curdlan, gum arabic, pullulan and other microbial polymers, polyvinylpyrrolidone, pectin, starch and its derivatives, gelatin, cellulose derivatives (including sodium cellulose sulphate, carboxymethyl cellulose, ethyl cellulose, hydroxypropyl methyl cellulose, hydroxyethyl cellulose, hydroxy propyl cellulose), synthetic and natural latex materials, casein, sodium silicate, polyvinyl alcohol, polyvinyl acetate, shellac, mono- and polyacrylates (including trimethylolpropane triacrylate); mono- and polymethacrylates (such as Paraloid B72 ethyl methacrylate copolymer), guar gum, Tragacanth gum, Konjac glucomannan and its derivatives;
cross-linking agents may include: organic and inorganic acids (such as formic, ascorbic, tannic, glycollic, gallic, acetic, salicylic, succinic, oxalic, citric, hydrochloric, periodic), glycerol, bases (including sodium hydroxide, ammonia, amines), organo silicates (including tetramethyl and tetraethyl orthosilicates), alkoxides of transition metals, organo-metallic compounds (including titanium butylate and zirconium propylate, and equivalent organometallic salts), calcium salts, potassium salts, zinc salts and other transition metal salts, silicones (including reactive silicones such as polymethylhydrogensiloxane), aldehydes (including glutaraldehyde, glyoxal and complex aldehydes such as cinnamoaldehyde and paraformaldehyde), vanillin, polybetaine, dextran sulphate, epichlorohydrin, natural cross-linkers (including genipin); amines (including ethylene diamine and ethylene triamine), glyceryl triacetate, triethyl citrate, triethyl phosphate, tripoly- or trimeta- or hexameta- or other cross-linking phosphates and phosphorus compounds, borates and other boron containing compounds;
dispersants/surfactants or surface tension adjusting substances may include: benzenesulphonic acid derivatives, trioctylphosphine oxide, polycarboxylic ammonium salts, various surfactants (including non-ionic, anionic, cationic, zwitterionic and neutral surfactants), such as carboxylate, ethoxylate, ester and amine oxide derivatives of sorbitan, laurate, stearate, oleate, glycerol, caprylic, caproic and higher chain length carboxylates, sucrose, glyceryl and polyglyceryl esters and fatty acid esters (including esters of lauric, myristic, palmitic, stearic, oleic, erusic and behenic acids), polymers which affect surface tension (including polyvinylpyrrolidone), inert superoleophilic metal powders and fibres such as stainless steel alloys, titanium and other refractory metal powders, fibres and flakes, and/or metal coated materials such as glass, ceramic and polymer particles, flakes and fibres;
gas generating agents may include ammonium and sodium bicarbonate;
photoinitiators may include: sodium benzoate, propyl p-hydroxybenzoate, methyl p-hydroxybenzoate, benzophenone (BP), 4,4′-bis(diethylamino)benzophenone (DEAB). 2-chloro-9H-thioxanthen-9-one (CTX), 1-chloro-4-ropoxy-9H-thioxanthen-9-one (CPTX), 2,2-dimethoxy-2-phenyl acetophenone (DMPA), 4-(dimethylamino)benzophenone (DMBP), 2-ethylanthraquinone (EA), 2-ethylhexyl-4-dimethylaminobenzoate (EDB), ethyl-4-dimethylaminobenzoate (EDMAB), 4-hydroxybenzophenone (4-HBP), 1-hydroxycyclohexyl phenyl ketone (HCPK), 2-hydroxy-4-methoxybenzophenone (HMBP), 2-hydroxy-4′-(2-hydroxyethoxy)-2-methylpropiophenone (HMMP), 2-isopropyl-9H-thioxanthen-9-one (ITX), 4-methylbenzophenone (MBP), 4,4′-bis(dimethylamino)benzophenone (MK) and 4-phenylbenzophenone (PBZ);
magnetic materials may include: nano- to micron-sized iron, nickel, cobalt and rare earth metals in spherical, flake or fibre form;
controlled release aids may include: shellac and shellac mixtures with glycerol or polypropylene glycol;
further auxiliary components which may be used individually or in combination with any other functional auxiliary components may include: pigments, photo- or heat- or pressure- or moisture- or gas-sensitive agents, pharmaceuticals, pesticides, micro-organisms, catalysts, chemicals which act as a deterrent, flavours, fragrances, cosmetic ingredients, nutritional or probiotic or neutraceutical ingredients (e.g. including lyophilised micro-organics), radioactive or pyrotechnic ingredients, density adjusting materials.

In practical embodiments the overall compositions, formulations and constituents of each of the first and the second compositions, or at least the constituents thereof other than the respective first and second reactive substances, may be selected independently of each other, for example depending on the chemical identity and reactive nature of the respective first and second substances.

In various embodiments of the invention, the first composition may be selected from a generally substantially aqueous or hydrophilic composition, or a substantially oily or hydrophobic composition, or an electrically conductive composition, or a substantially electrically non-conductive composition, or possibly even a substantially electrically semiconductive composition. Likewise, but independently of the first composition, in various embodiments of the invention the second composition may also be selected from a generally substantially aqueous or hydrophilic composition, or a substantially oily or hydrophobic composition, or an electrically conductive composition, or a substantially electrically non-conductive composition, or possibly even a substantially electrically semiconductive composition.

In some such embodiments of the invention, at least one of, optionally either or each or both of, the first composition (including the first reactive substance) and/or the second composition (comprising the second reactive substance) may include one or more auxiliary component substances, which may be either chemically inert or reactive so as to be involved in the chemical reaction between the first and second reactive substances. For example, a chemically inert auxiliary substance may serve to alter or adjust one or more physical properties of the respective composition in which it is incorporated, e.g. an inert powder or particulate material to adjust viscosity, or a buffering agent to adjust or maintain a desired pH, or magnetic particles to enable product bodies to be manipulated using a magnetic field in order to move them into a location in the dielectric medium at which they may be more readily collected and/or recovered, or possibly even electrically conductive particles, filaments, tubes or flakes (e.g. of a nano-metal or -metal alloy or a non-metal) to adjust the electrical conductivity characteristics of the composition into which it is incorporated. On the other hand, a chemically reactive auxiliary substance may serve as a co-reactant, initiator or catalyst or other promoter of the main chemical reaction between the first and second reactive substances. As examples of suitable such auxiliary substances of the above types may be mentioned any of the following: any known reaction catalysts, initiators or photo-initiators, a nano-metal substance (e.g. particles, filaments, tubes or flakes of copper, silver or nickel (e.g. from Promethean Particles Ltd) (or other catalytic metal), or non-metal filaments, tubes or flakes (e.g. graphene from ArcelorMittal)), having a particle size in the range of from about 1 up to about 500 nm, e.g. from about 1 or 2 or 3 up to about 6 or 20 or 50 or 100 or 250 nm, or optionally a polymer-(e.g. PVP)-stabilised nano-copper [or other metal] material (e.g. from Promethean Particles Ltd)).

In the practising of embodiments of the invention the first and second substances for reaction together may be selected from a wide variety of species in a wide variety of industrial, chemical or pharmaceutical fields. For example, some embodiments of the invention in its various aspects may be applicable to the synthesis of various chemical compounds, e.g. certain polymers formed by polymerisation reactions between at least one monomer (as one of the first or second substances) and a polymerisation catalyst or photo-initiator (e.g. UV photo-initiator) or other reactant or another monomer (as the other of the first or second substances), or certain cross-linked products obtained by cross-linking certain polymeric molecules (as one of the first or second substances) using a cross-linking agent (as the other of the first or second substances). Other embodiments of the invention in its various aspects may be applicable to the synthesis of pharmaceutical actives, pesticides, herbicides, flavours or fragrances, food products or food additives, drink additives, neutraceuticals, confectionery, additives for use in various industrial manufacturing processes and applications, as well as to the carrying out of chemical reactions for the production of certain synthetic fibres, alloys, electronic materials, explosives, pyrotechnic substances, and nanomaterials, as well as possibly other kinds of end products that involve some kind of chemical reaction between reactive reactant or reagent species. Yet other embodiments of the invention in its various aspects may be applicable to the production or formation—through merely physical interactions of the first and second bodies—of novel physical forms, configurations or structures of e.g. pharmaceutical actives or products, pesticides, herbicides, flavours or fragrances, food products or food additives, drink additives, neutraceuticals, confectionery, additives for use in various industrial manufacturing processes and applications. certain synthetic fibres, alloys, electronic materials, explosives, pyrotechnic substances, or nanomaterials.

Embodiments of the invention are especially applicable to the carrying out of electrostatically-induced chemical or physical reactions between species under anaerobic or aseptic conditions. Accordingly, in some embodiments of the invention the various methods according to aspects of the invention may be carried out under conditions in which free oxygen is substantially absent from/in the said dielectric medium in which the charged first and second bodies are formed. For example, such anaerobic conditions may also be useful in the production of certain pharmaceuticals, or in the reaction of certain nano-materials to form new compounds, and also of certain alloys, where surface energies may prohibit reaction under normal atmospheric conditions due to oxidation or adsorption of gases or where high voltage discharging promotes an unwanted reaction. And in some other embodiments of the invention the various methods according to aspects of the invention may be carried out under conditions in which one or more pathogenic substances is/are substantially absent from/in the said dielectric medium in which the charged first and second bodies are formed.

In many practical embodiments of the invention the formation of each respective one(s) of the first and second bodies may be effected substantially simultaneously with the application thereto of the respective electric charges of opposite polarity that are applied to each respective body species. Alternatively, in other embodiments, either the first or the second body(ies), or both thereof, may have their respective electric charge(s) applied thereto subsequent to the actual formation of the respective body(ies) themselves.

In many practical embodiments of the apparatus of the invention, the first means for forming the said one or more first bodies comprising the first substance and applying thereto an electric charge of a first polarity may comprise a first nozzle device and a first charging means. The first nozzle device and first charging means may in some embodiments be provided in combination with one another in a single substantially unified first delivery means which serves both purposes, or alternatively in other embodiments the first nozzle device and the first charging means may be provided as discrete components of a two-stage or two-element first delivery means of which each discrete element effects a respective one of the first body-forming and the first body-charging functions.

Likewise, but independently of the construction and operation of the first means, the second means for forming the said one or more second bodies comprising the second substance and applying thereto an electric charge of a second polarity may comprise a second nozzle device and a second charging means. The second nozzle device and second charging means may in some embodiments be provided in combination with one another in a single substantially unified second delivery means which serves both purposes, or alternatively in other embodiments the second nozzle device and the second charging means may be provided as discrete components of a two-stage or two-element second delivery means of which each discrete element effects a respective one of the second body-forming and the second body-charging functions.

Thus, in certain embodiments of the invention the manner in which either or each of the first and second nozzle devices, together with their associated or integrated respective first and second charging means, form and eject a respective one or more of (e.g. a group or cluster or cloud or series or sequence or chain of a plurality of) the respective oppositely charged first and second bodies may involve an electrohydrodynamic comminution process or phenomenon. Thus, such embodiments of the invention may be characterised by the respective first and second bodies being formed and ejected from their respective nozzle and charging devices by virtue of an electrohydrodynamic comminution process or phenomenon.

However, in many other embodiments of the invention the manner in which either or each of the first and second nozzle devices, together with their associated or integrated respective first and second charging means, form and eject a respective one or more (e.g. a group or cluster or cloud or series or sequence or chain or cluster of a plurality of) of the respective oppositely charged first and second bodies may involve a more straightforward droplet- or particle-formation and release (or emission) process or step. Thus, such embodiments of the invention may be characterised by the respective first and second bodies being formed and ejected from their respective nozzle and charging devices by virtue of a droplet- or particle-formation and release (or emission) process or step.

In the context of either of the embodiment species in either of the preceding paragraphs, once they have been so formed and ejected from their respective nozzle devices, the respective charged first and second bodies may thus become attracted together in respective oppositely charged pairs thereof by virtue of electrostatic attraction, as governed by Coulomb's Law, thereby resulting in a joining or merging or combining together—optionally with whole or partial electric neutralisation—of the charged first and the second bodies so as to form any of the above-defined one or more product bodies, wherein the one or more product bodies comprise both the first and the second substances, which first and second substances are thereby able to interact and/or react together within the one or more product bodies. Additionally or alternatively, the electrostatic attraction and coming together (or joining or merging or combining together) of the pairs of first and second bodies may, rather than inducing a chemical reaction between the first and second substances therein, be such as to induce a merely physical interaction or reaction between, or reconfiguration of, the first and second bodies, whereby the product bodies resulting therefrom are of a novel physical form, configuration or structure, rather than containing a chemically novel product.

In some such embodiments either or each of the first and/or second nozzle device(s) may comprise a respective outlet or mouth connected to a respective feed tube, conduit, pipe, capillary or channel which is in fluid communication with a respective reservoir containing a supply of the respective first or second composition comprising or including the respective first or second reactive substance.

In embodiments, the respective feed tube, conduit, pipe, capillary or channel of each of the first and second nozzle devices may be formed of any suitable material, which may be either electrically conductive or non-conductive. For example, the respective feed tube, conduit, pipe, capillary or channel of each of the first and second nozzle devices may be non-metallic (e.g. of pultruded electrically conductive carbon fibre or an electrically non-conductive glass material or an electrically conductive or non-conductive polymer) or may alternatively be made of a suitable metal or metal alloy (e.g. a steel or an inert metal or metal alloy). The latter may be particularly useful in embodiments where electrical connection of the respective charging means is desired directly to the outlet/mouth of the respective nozzle device.

However, in certain embodiments, especially where semiconducting fluid first and/or second reactive substances are employed, the material of the respective feed tube, conduit, pipe, capillary or channel of each of the first and second nozzle devices may be of a high electrical conductivity, since with such semiconducting fluid reactive substances charge is transferred from the connection point with the high voltage source through the fluid to the respective nozzle, but if the fluid substance is insufficiently conductive (e.g. with specific resistivity of >10-6 ohm·cm) then an electrically conducting nozzle device may be required.

The outlet or mouth of each respective nozzle device may be formed with any suitable size, shape and configuration, especially size and cross-sectional shape, e.g. as may be desired or appropriate for forming the respective first or second bodies so as to have a particular desired body size and body shape or physical form. For example, the outlet or mouth of each respective nozzle device may in cross-section be generally substantially circular, elliptical, rectangular, square, polygonal, or slot- or slit-shaped. Furthermore, in certain embodiments, if desired or appropriate, the outlet or mouth of each respective nozzle device may be fitted or provided (or formed) with an e.g. silicone duckbill or other exit flow control feature or attachment, such as a valve, e.g. a cross-slit, belleville, X-fragm, or dome elastomeric self-closing valve. Moreover, the outlet or mouth of each respective nozzle device may have a size, especially an internal width (e.g. an internal diameter), appropriate to a desired size of the respective bodies to be formed thereby. By way of example, an outlet/mouth internal width or diameter in the range of from about 0.01 or 0.02 or 0.03 or 0.04 or 0.05 or 0.06 or 0.07 or 0.08 or 0.09 or 0.1 or 0.2 or 0.3 or 0.4 or 0.5 mm up to about 2 or 3 or 5 or 6 or 7 or 8 or 9 or 10 mm may be suitable in many practical embodiments.

In practising embodiments of the invention, the resulting size or width of the first and/or second charged bodies formed by the respective outlets or mouths of the respective nozzle devices may be related to the respective outlets/mouths' internal width or diameter, as defined generally above. Typically, for example, smaller nozzles may give rise to narrower body size distributions compared with larger nozzles, under typical electrospraying regimes. However, in the creation of large bodies of especially viscous liquid first or second compositions the normal relationships between nozzle widths/diameters and body sizes may no longer apply. In general, therefore, nozzle widths/diameters of around 0.5 mm (internal diameter) for low viscosity liquids and of around 1-2 mm (internal diameter) for large bodies towards the upper end of the above-defined overall ranges may be suitable. The smaller sized nozzles may give rise to bodies of a relatively wide size distribution when operated in electrospray mode, and the larger sized nozzles may give rise to bodies of a relatively narrow size distribution when operated in non-electrospray or electrostatic attraction mode. In the case of the larger sized nozzles in particular, the nozzle tip may if desired or appropriate be fitted or provided (or formed) with any of the above-mentioned e.g. silicone duckbill or equivalent exit control feature or attachment, such as a cross-slit, belleville, X-fragm, dome elastomeric self-closing valve.

In some embodiment forms the respective outlets or mouths of the first and the second nozzle devices may be of different, possibly even substantially different, shapes and/or sizes and/or widths compared with one another, e.g. so that they may for instance form and deliver first and second bodies of substantially different shapes and/or sizes and/or widths. However, in other embodiment forms the respective outlets or mouths of the first and the second nozzle devices may be of substantially or approximately equal shapes and/or sizes and/or widths compared with one another, e.g. so that they may for instance form and deliver first and second bodies of substantially equal or comparable shapes and/or sizes and/or widths.

Thus, and as already defined above, in many practical embodiments of the invention the first and second bodies comprising, respectively, the first and second compositions comprising the first and second reactive substances may have typical body widths or diameters in the range of from about 0.01 or 0.02 or 0.03 or 0.04 or 0.05 or 0.06 or 0.07 or 0.08 or 0.09 or 0.1 or 0.2 or 0.3 or 0.4 or 0.5 mm up to about 2 or 3 or 5 or 6 or 7 or 8 or 9 or 10 or 15 or 20 mm.

Furthermore, in some alternative embodiment forms it may even be possible for the outlet or mouth of each respective nozzle device to be subdivided or split into a plurality of individual discrete sub-outlets or sub-mouths, e.g. pores or apertures, which are able to form a plurality of individual discrete sub-bodies of the respective first or second composition in close proximity to each other in a single given delivery operation.

In some embodiments the or each respective reservoir may be provided with, or include, feed or injection or pump means, e.g. a positive displacement or syringe-type device or a piston-in-cylinder device or a peristaltic device, for delivering or forcing the respective first or second composition to the respective nozzle device's outlet/mouth in a controlled manner, especially at a particular predetermined desired flow rate or speed or in a particular predetermined dose amount or volume per given delivery operation, step or stage. The or each respective such feed or injection or pump means may be operated and/or controlled either manually or under the control and operation of an automated control system, such as an electronic or computer/software-controlled control system.

In some example embodiments suitable flow rates by which the respective first or second composition may be delivered from its respective nozzle device may typically be in the range of from about 0.1 or 0.15 or 0.2 or 0.25 or 0.3 or 0.4 or 0.5 or 0.6 or 0.7 or 0.8 ml/min up to about 5 or 6 or 7 or 8 or 9 or 10 ml/min, optionally from about 0.1 or 0.15 or 0.2 or 0.25 or 0.3 or 0.4 or 0.5 or 0.6 ml/min up to about 1 or 1.5 or 2 ml/min. By way of example, a flow rate of around 0.2 to 0.3 ml/min may be suitable for use with a 0.6 mm internal diameter nozzle device for the formation of relatively large first or second bodies (e.g. with diameters in the region of about 1-5 mm), whereas a flow rate of around 0.3 to 0.5 ml/min may be suitable for use with a 0.6 mm internal diameter nozzle device for the formation of relatively small first or second bodies (e.g. with diameters in the region of about 0.05 or 0.1-1 mm). In any given example practical system the individual flow rates by which each of the first and the second compositions may be delivered into the dielectric medium may be selected and controlled in order to best suit the rheological characteristics of the respective first and second compositions, which optimisation will be within the ordinary skill of the person skilled in the art.

In some embodiments it may be desirable to employ a valve device at the or each nozzle device's outlet or mouth, examples of which have already been mentioned above. Thus, and in the case of certain embodiments, it may be necessary or desirable to employ concentrated first and/or second reactive compositions which have a high viscosity, which may necessitate a relatively large bore nozzle device (e.g. approx. 1 or 2 or 3 mm in internal diameter). At a certain reactive composition liquid viscosity the electrostatic force may be insufficient to overcome the viscous force holding the liquid in place at the nozzle outlet/mouth, so the charge/unit mass ratio may decrease with increasing body size, because the body mass increases with the cube of its radius whereas the body charge increases only as a^{3/2}. Without modification, therefore, in such circumstances a pumped reactive composition may discharge as a continuous string or fibre into the dielectric fluid medium, and once they are sufficiently long the fibres from plural such nozzles may meet and thereby short-circuit the voltage power supply (e.g. transformer). Desirably, therefore, a silicone duckbill valve or equivalent shut-off valve made from a low surface energy deformable polymer, may be attached to the or each nozzle tip. (Such valves are currently manufactured as non-return valves for use in petrol containing reservoirs.) Fluid may be pulsed on and off by a delivery pump using a programmable timing device or equivalent mechanical switching device, so that each pulse delivers one drop mass of the relevant reactive composition. When the pump is switched off and not delivering fluid, the duckbill or other valve is forced to shut due to elastic forces in the rubber body of the valve. This reduces the cross-section of liquid which is attached to liquid in the delivery nozzle device such that the electrostatic force can overcome the viscous force and cause a body to detach from the nozzle outlet/mouth and be attracted to a body of opposite polarity in the dielectric medium.

In certain embodiments of the invention, one or more of the first and/or second nozzle devices may be provided with heating means, for heating the respective first or second composition as it approaches the outlet or mouth of the respective nozzle device and exits therefrom, for the purpose of locally or temporarily reducing the viscosity of that respective composition and thereby altering its body-forming properties. For example, by locally or temporarily reducing the viscosity of that respective composition in this manner (e.g. using an EnFlow IV fluid heater device manufactured by GE Healthcare), it may be possible to promote or encourage the formation of discrete bodies of that respective composition (e.g. in the form of discrete separate droplets) which tend to detach themselves more easily from the outlet/mouth of the respective nozzle device, instead of possibly tending instead to coalesce into respective bodies of that respective composition in the form of fibres or threads or chains before being brought together with the charged bodies of the other respective composition.

In some embodiments either or each of the first and/or second charging means, which apply the necessary electric charges of opposite polarities to the formed first and second bodies, may comprise any suitable respective electric charge source(s) or charge delivery device(s). Suitable such charge delivery devices may include a charging plate or ring or other suitable charge-transfer element connected to the respective nozzle device. Such electric charge source(s) and/or delivery device(s) may be mains powered or may be powered from a battery source. In the case of an AC power source (e.g. from the mains), any suitable rectifier apparatus or device may be employed to obtain a desired DC low current output, for generating the required final voltage and applying it to the apparatus of the invention.

In some embodiments a common power supply apparatus may be employed for generating and delivering both the positive and the negative polarity electric charges to the formed first and second bodies, although in other embodiments independent power supply apparatuses may be provided for generating and delivering each respective electric charge to the formed first and second bodies separately from and independently of each other.

In many practical embodiments suitable electric charge generators or sources may supply electric voltages from about 1 kV up to about 20 or 30 kV, especially with only micro- to milliamp current drain (noting in particular that the apparatus in question may be driven primarily by charge, not by current). In some typical example embodiments, however, electric charge generators or sources supplying electric voltages from about 500 V or 2 or 3 kV up to about 10 or 15 kV may be commonly employed, especially in the context of bringing about electrostatic attraction and bringing together of relatively large first and/or second bodies (e.g. with diameters in the region of about 1-5 mm). On the other hand, in other typical embodiments, in particular in the context of bringing about electrostatic attraction and bringing together of relatively small first and/or second bodies (e.g. with diameters in the region of about 0.05 or 0.1-5 or 6 mm), electric charge generators or sources supplying electric voltages from about 1.5 or 5 or 6 kV up to about 20 or 30 kV may be more suitable. By way of example, in some currently implemented example embodiment systems first and/or second bodies with diameters/widths in the region of about 1-5 mm may be used in combination with electric charge generators or sources supplying electric voltages of around 1.5-6 kV. Of course, other body sizes and associated voltages may be employed in other example embodiments. Specific practical examples of such electric charge generator or source apparatuses or devices that may be suitable for use in embodiments of the invention will be well-known laboratory equipment and widely available to persons skilled in the electrochemical arts.

In the practising of some embodiments of the invention the voltage applied by the first charging means to first bodies of the first substance may be substantially or approximately the same as the voltage applied by the second charging means to second bodies of the first substance. In this manner, in such embodiments the respective pairs of charged first bodies and charged second bodies may have substantially equal yet opposite electric charges applied to them. However, in other embodiments the voltage applied by the first charging means to the first bodies of the first substance may be different from, or indeed significantly different from, the voltage applied by the second charging means to second bodies of the first substance. In the latter manner, in such embodiments the respective pairs of charged first bodies and charged second bodies may have significantly unequal and opposite electric charges applied to them.

Moreover, in carrying out various embodiments of the invention, when the charged first and charged second bodies are brought together (and optionally merged or united together) by virtue of the resulting electrostatic attraction between the respective pairs thereof, the electric charges applied to the respective first and second bodies may or may not substantially cancel each other out, or become neutralised, in the formation of the respective product bodies, following that bringing together (and/or merging or uniting together) of the first and second bodies. Thus, in some embodiments of the invention the respective product bodies resulting from the bringing together (and/or merging or uniting together) of the first and second bodies may have or exhibit a residual electric charge of substantially 0 V, whereas in other embodiments the respective product bodies may have or exhibit a positive or a negative electric charge anywhere up to e.g. 1%, 5%, 10%, 20%, 30%, 40%, 50%, 60%, 70%, 80%, or 90% of the electric charge initially applied to either of the first or second bodies.

In accordance with the present invention the one or more charged first bodies and the one or more oppositely charged second bodies are each formed in the dielectric medium, especially a dielectric medium comprising the one or more dielectric fluids or pseudo-fluids. Thus, in practical embodiments of the invention in its various apparatus and method aspects, the respective outlets or mouths of each respective nozzle device that delivers or ejects its respective first or second body(ies) may be submerged in the dielectric medium, optionally submerged in and at least one of the one or more dielectric fluids or pseudo-fluids that form(s) the dielectric medium.

Thus, in embodiments in which the dielectric medium comprises just a single dielectric fluid/pseudo-fluid, both of the respective outlets or mouths of the respective nozzle devices that deliver or eject their respective first and second body(ies) may be submerged in the said dielectric fluid/pseudo-fluid forming the dielectric medium, whereby the charged first bodies are formed in the same phase, layer or volume of the dielectric medium as are formed the charged second bodies.

However, in embodiments in which the dielectric medium comprises a plurality of different dielectric fluids/pseudo-fluids, especially at least one hydrophobic (or oliophilic) dielectric fluid/pseudo-fluid and at least one hydrophilic dielectric fluid/pseudo-fluid, which form respective distinct separate phases or layers in the overall dielectric medium, each of the respective outlets or mouths of the respective nozzle devices that deliver or eject their respective first and second body(ies) may be submerged in either (a) the same discrete phase or layer of a given one of the dielectric fluids/pseudo-fluids in the overall dielectric medium, or (b) in respective different ones of the discrete phases or layers of the dielectric fluids/pseudo-fluids in the overall dielectric medium, whereby the charged first bodies are formed in a different discrete phase or layer of the dielectric medium from the discrete phase or layer thereof in which are formed the charged second bodies.

In various practical embodiments of the invention, the first and second nozzle devices may be mounted or supported in the container (containing the dielectric medium) in any desired or suitable orientation and relative positioning or configuration. In particular, the first and second nozzle devices may be mounted or supported relative to each with any suitable distance separation (especially distance separation at their respective free ends/tips) and/or at any suitable angle separation relative to each other (i.e. the angle subtended by the longitudinal axes of the two nozzle devices, relative to each other).

For example, a suitable or optimum distance separation of the first and second nozzle devices (especially the distance separation at their respective free ends/tips) may be selected depending primarily on the breakdown voltage/potential of the dielectric medium between them, so as to avoid exceeding a threshold voltage/potential difference at which short-circuiting between the two nozzles and/or charging apparatuses/devices occurs. In any practical example embodiment a most suitable or optimum distance separation of the first and second nozzle devices (especially the distance separation at their respective free ends/tips) may further be selected depending on the charge carrying properties of the first and second reactive substances in the first and second bodies. In many practical embodiments such a distance separation of the first and second nozzle devices (especially the distance separation at their respective free ends/tips) may be in the range of from about 3 or 4 or 5 or 6 or 7 or 8 mm up to about 10 or 12 or 15 or 20 or 25 or 30 mm. Alternatively or additionally, for example, a suitable or optimum angle separation of the first and second nozzle devices relative to each other (i.e. the angle subtended by the longitudinal axes of the two nozzle devices, relative to each other) may be in the range of from about 45° up to about 120°, optionally from about 50 or 60° up to about 90 or 100 or 110°.

In certain embodiments, in particular those in which the first and second substances being reacted together may lead to the possible formation of chains or series or clumps/clusters of interconnected product bodies, it may also be desirable to select or adjust such a distance separation of the first and second nozzle devices and/or such an angle separation thereof such that any risk of such chains or series or clumps/clusters building up and forming a bridge and thus themselves short-circuiting the two nozzles and/or charging devices may be lowered or ameliorated or even removed. Alternatively or additionally, in certain embodiments, as a means of preventing or reducing the tendency for formation of such chains or series or clumps/clusters of product bodies that could lead to such bridging and thus short-circuiting, the apparatus may further include means—e.g. a separate pumping or recirculation device—for providing an auxiliary flow (e.g. laminar flow, which may be in a generally upward or downward direction) of a dielectric fluid/pseudo-fluid (which may the same as or different from the dielectric fluid/pseudo-fluid in which the nozzle(s) is/are located) within the overall dielectric medium away from the region at which the nozzles' outlets/mouths are closest together.

Although in many embodiments of the invention the first and second nozzle devices may be constructed and arranged so as to be separated from each other (e.g. by a suitable distance separation and/or angle separation as defined above), in certain other embodiments of the invention the first and second nozzle devices may be constructed and configured or arranged so as to be concentric or co-axial with respect to one another, e.g. such that one nozzle device at least partially surrounds the other nozzle device. In this manner one of the first and second bodies may each be formed so as to be in the form of a core body which is surrounded or encapsulated or enshrouded by the other of the first and second bodies which is in the form of a hollow or shell body. In such embodiments the associated respective feed tubes, conduits, pipes, capillaries or channels which are each in fluid communication with the respective reservoirs containing the supplies of the respective first and second compositions (comprising or including the respective first and second reactive substances) may if desired be likewise constructed or arranged concentrically or co-axially with respect to one another, or alternatively may be simply arranged in the same general fashion as in other non-concentric/non-co-axial embodiments and be connected to their respective nozzle device via an appropriate route and at an appropriate location adjacent their concentrically or co-axially arranged respective outlets/mouths.

Although in many embodiments of the invention two (i.e. first and second) nozzle and associated or integral charging means may be employed, in certain other embodiments of the invention one or more third or additional nozzle and associated or integral charging means may be provided, for forming and delivering one or more third or additional charged bodies into the dielectric medium, for reaction with either of, or even both of, the first or second charged bodies.

Such third or additional charged bodies may comprise a third composition comprising a third reactive substance, for reaction with either the first or the second substance or an intermediate product of the first and second substances once they have already reacted together. Thus, in some such embodiments the third reactive substance may be a different species or substance from both the first and the second substances—for example the third reactive substance may be a reaction catalyst or photo-initiator for promoting a chemical reaction (e.g. a polymerisation or cross-linking reaction, or alternatively some other chemical reaction) between the first and the second reactive substances. However, in certain other such embodiments it may be possible for the third reactive substance to be the same as either of the first or the second substances, e.g. in the case where further units of a polymer material are desired to be added to a growing chain-like molecule, or perhaps even where cross-linking is desired to be effected once a basic polymeric structure has been created.

Where it is provided, any third or respective additional reactive substance may be provided in its, or its respective, third or additional composition optionally together with one or more other, auxiliary components of the third or additional composition, e.g. one or more solvents, co-solvents, diluents, emulsifiers, stabilisers, viscosity modifiers, preservatives, pH adjusting agents, etc, which auxiliary components may be selected independently of any corresponding components that may be present in the first and/or the second compositions comprising the first and second reactive substances.

In such embodiments where third or additional charged bodies of a third reactive substance are formed and delivered into the system, the electric charge applied to such third bodies may be of the same polarity as the charge originally applied to either the first or the second bodies, and it may be applied through generation of a different, optionally a significantly different, voltage as is used to charge the first and the second charged bodies. In particular, a third or additional charged body species may be introduced into the system in a case where a residual positive or negative charge is left on the product bodies of the reacted first and second bodies and the third or additional charged body species may in that case have applied thereto an electric charge of a polarity opposite to that of the aforementioned residual charge.

In the case of embodiments including an above-defined third or additional nozzle and associated or integral charging means for delivering into the system one or more third bodies, in the context of embodiments as defined above in which the first and second nozzle devices are constructed and configured or arranged so as to be concentric or co-axial with respect to one another (in order to form a core-within-a-shell type of first-and-second bodies structure/arrangement), in such embodiments it may be possible for the third or additional nozzle likewise to be constructed and configured or arranged so as to be concentric or co-axial with respect to the first and second nozzle devices, so that the third nozzle forms and delivers third bodies which are themselves of the nature of an additional layer or coating or casing or outer shell encapsulating or enshrouding the core-within-a-shell type of first-and-second bodies arrangement. Such a concentric or co-axial arrangement of such a third nozzle device may be useful in certain specialist embodiments for enabling an especially large (e.g. >5 mm diameter/width) core-within-a-shell type of first-and-second bodies to be exteriorly supported, strengthened or hardened, especially if it is somewhat “floppy” or soft. As just one illustrative example of such a three-component outer-casing+core-within-a-shell structure/arrangement used in an example embodiment of the invention there may be mentioned a combined structure comprising a soft chocolate/vanillin/meta- or pyro-phosphate core, a chitosan/pullulan in acetic acid solution (phosphate and vanillin being chitosan cross-linkers), and a shellac outer casing (shellac polymerises in contact with acid), all in a dielectric fluid medium comprising paraffin or silicone oil. As another illustrative example of such a three-component outer-casing+core-within-a-shell structure/arrangement used in another example embodiment of the invention there may be mentioned a combined structure comprising a core of chitosan solution in acetic acid mixed with a pyrethroid and an ethanol solution of shellac and/or hydroxypropyl methyl cellulose and/or ethyl cellulose shell forming materials in a dielectric fluid medium of paraffin oil or silicone oil. In this case the shellac polymerises and precipitates to form a hard shell in contact with the acid medium of the core forming a capsule.

In various practical embodiments of the invention, the location in the container (containing the dielectric medium) at which are mounted or supported the first and second nozzle devices (or, rather, at least the outlets or mouths thereof) may depend on the relative densities of the dielectric medium and the relevant product bodies following reaction of the first and second bodies. For instance, in embodiments in which the dielectric medium has a density greater than the density of the relevant product bodies, it may be convenient or desirable for the first and second nozzle devices to be located in a lower region of, i.e. adjacent or in the vicinity of the bottom or lowermost portion of, the container containing the dielectric medium. In this manner, the lower-density product bodies may naturally tend to float upwards within the dielectric medium and into an upper region of the dielectric medium in the container, from where their separation and collection may be more readily carried out. Conversely, in embodiments in which the relevant product bodies have a density greater than the density of the dielectric medium, it may be convenient or desirable for the first and second nozzle devices to be located in an upper region of, i.e. adjacent or in the vicinity of the top or uppermost portion of, the container containing the dielectric medium. In this manner, the higher-density product bodies may naturally tend to sink downwards within the dielectric medium and into a lower region of the dielectric medium in the container, from where their separation and collection may be more readily carried out.

In certain embodiments of the invention—namely those in which the dielectric medium comprises a plurality of different dielectric fluids/pseudo-fluids, especially at least one hydrophobic (or oliophilic) dielectric fluid/pseudo-fluid and at least one hydrophilic dielectric fluid/pseudo-fluid which form respective distinct separate phases or layers in the overall dielectric medium, and the dielectric fluids/pseudo-fluids are selected such that a lighter, e.g. silicone oil, phase is used in combination with a heavier, e.g. aqueous, phase (such as in the use of deionised water with a mixture of methylene chloride and silicone oil, to name just one example of such plural dielectric fluids, although alternatively if the silicone oil is mixed with methylene chloride the resulting mixture is denser than the aqueous water phase, so the relative positions of the two phases may in that case be reversed)—the outlet/mouth of a nozzle device delivering an oil-based first or second body reactant substance may be located in the aqueous dielectric fluid/pseudo-fluid phase and the outlet/mouth of a nozzle device delivering an aqueous first or second body reactant substance may be located in the silicone oil dielectric fluid/pseudo-fluid phase, with both nozzle devices being mounted or supported relative to each other such that oil-based charged first or second bodies formed in the aqueous dielectric phase may be electrostatically attracted to aqueous charged first or second bodies formed in the oil dielectric phase and may react together before their product bodies either rise to the surface of the aqueous phase or sink therein, depending on the density of the resulting product bodies.

As another example of such a system as in the preceding paragraph, in the case of a two-component dual-phase/layer dielectric fluid medium comprising a nut oil (i.e. lighter than water), such as sweet almond oil, and deionised water, aqueous charged first bodies delivered into the almond oil phase may be electrostatically attracted to oil-based charged second bodies formed in the water phase, thereby bringing about or facilitating an electrostatically-induced chemical reaction between the respective first and second reactant substances at the oil/water interface, as a result of which the thus-formed product bodies may either fall, rise or remain at the oil/water interface, depending on the product body density.

In some embodiments of the invention, the basic method of bringing together the one or more first bodies comprising the first reactive substance with the one or more second bodies comprising the second reactive substance, whereby the first and second substances are electrostatically induced or facilitated to chemically and/or physically react together, may be carried out in any number of reaction stages or cycles, such that any product bodies resulting from the merging of the first and second bodies and/or from the said reaction may either be (i) collected or recovered once formed, and then used for any desired onward purpose external to the system, or optionally (ii) collected or recovered once formed, and then recycled by being passed through the system one or more further times, e.g. by using them—either in their as-produced form or following a reformulation or intermediate treatment thereof—as one of the “first body” species or “second body” species in one or more subsequent runs or cycles of the method of the invention being carried out.

Within the scope of this specification it is envisaged that the various aspects, embodiments, examples, features and alternatives, and in particular the individual constructional or operational features thereof, set out in the preceding paragraphs, in the claims and/or in the following description and accompanying drawings, may be taken independently or in any combination of any number of same. For example, individual features described in connection with one particular embodiment are applicable to all embodiments, unless expressly stated otherwise or such features are incompatible.

### BRIEF DESCRIPTION OF THE DRAWINGS

Various embodiments of the present invention in its various aspects will now be described in detail, by way of example only, with reference to the accompanying drawings, in which:

FIG. 1 is a schematic sectional view of a reaction apparatus according to one embodiment of the invention;

FIG. 2 is a schematic sectional view of a modified form of reaction apparatus according to another embodiment of the invention, which is similar to that of FIG. 1 but in which a modified form of nozzle arrangement is employed;

FIG. 3 is a schematic sectional view of the lower region of another reaction apparatus according to yet another embodiment of the invention, in which two discrete dielectric fluids are employed as the dielectric medium;

FIG. 4 is a schematic sectional view of the lower region of another reaction apparatus according to still yet another embodiment of the invention, in which the nozzle arrangement is reversed in orientation and a pumping system is employed to recirculate dielectric fluid between lower and upper regions of the reaction container, for improved stability of product bodies produced between the two delivery nozzles;

FIG. 5 is a photograph of an example of a trial laboratory set-up of the apparatus shown schematically in FIG. 1, as used for conducting many of the working Example procedures as described in the working Examples hereinbelow;

FIG. 6 is a close-up photograph of the pair of composition delivery tubes and nozzles that are used to form the charged bodies of the first and second substance compositions in the apparatus of FIG. 5;

FIG. 7 is a graph showing the voltage calibration curve use in the calibration of the high voltage transformer used in certain ones of the working Examples described hereinbelow;

FIG. 8 is a side-on photograph of an alternative form of reaction vessel, in the form of a tube, embodying apparatus according to yet another embodiment of the invention;

FIGS. 9(a), 9(b) & (9c) are photographs of representative examples of discrete charged droplets containing first and second substances formed in accordance with an embodiment of the invention using the apparatus of FIG. 5 and in the process of, respectively, being formed (FIG. 9(a)), being brought together and merging (FIG. 9(b)) and undergoing reaction having been united (FIG. 9(c));

FIG. 10 is a schematic sectional view of a more advanced form of reaction apparatus according to yet another embodiment of the invention, in which a further modified form of nozzle arrangement is employed that produces concentric bodies of the first and second substance compositions;

FIG. 11 is a schematic sectional view of another more advanced form of reaction apparatus according to yet another embodiment of the invention, in which an additional third nozzle is employed for forming and delivering into the reaction system one or more third bodies comprising a third substance composition and charged with an electric charge different in polarity from that of a residual charged body resulting from the reaction between the first and second bodies; and

FIGS. 12(a) & 12(b) are photographs of two example species of microcapsule-type products produced by use of a prototype embodiment apparatus according to the invention, as described in the working Examples hereinbelow.

### DETAILED DESCRIPTION OF EMBODIMENTS

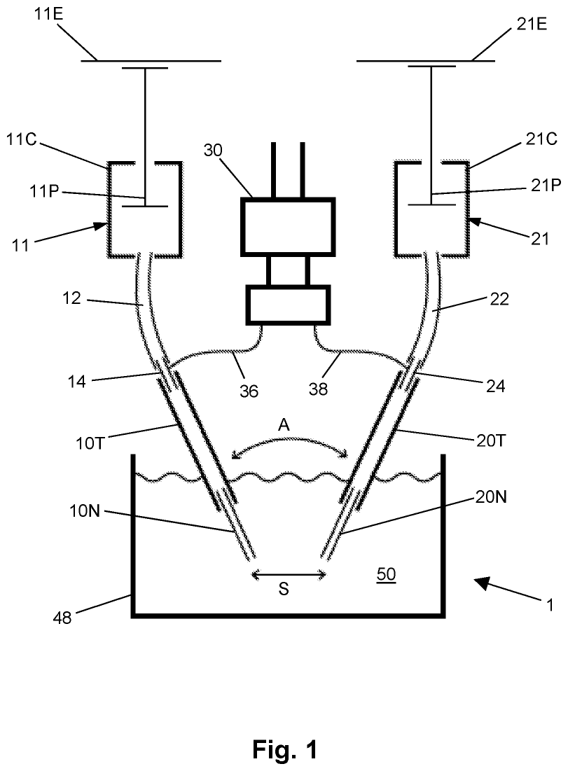

FIGS. 1 to 4 show schematically various forms of reaction apparatus in accordance with various embodiments of the invention, any of which may be used for the carrying out of a wide range of electrostatically-induced chemical and/or physical reactions between first and second reactive substances, in a wide variety of chemical, pharmaceutical or other fields.

Referring firstly to FIG. 1, this shows schematically a basic set-up of reaction apparatus 1 according to one embodiment of the invention, in which a rectangular-sectioned container 48 contains a dielectric fluid medium 50, which forms the reaction medium in which is carried out a desired electrostatically-induced chemical reaction between charged first and second bodies (not shown in the FIG.), which contain respective first and second reactive substances, which are delivered into the reaction medium via respective nozzles 10N, 20N. The nozzles 10N, 20N are each in the form of a slender cylindrical tube or capillary, e.g. made from carbon fibre or a polymer (or possibly even a metal or metal alloy), with an outlet or mouth with an internal diameter typically in the range of from about 0.1 (or possibly about 0.4 or 0.5) up to about 2 mm. The dielectric fluid medium 50 may be either an oil (i.e. hydrophobic) liquid phase, e.g. a silicone oil (or any of the other hydrophobic or oily liquid substances disclosed herein) or an aqueous (i.e. hydrophilic) liquid phase, e.g. deionised water oil (or any of the other hydrophilic liquid substances disclosed herein). The identity of the dielectric fluid medium 50 may in principle be selected from any of those exemplified hereinabove.

Each nozzle 10N, 20N is mounted into a respective feed tube or pipe 10T, 20T which is itself connected in fluid communication, via a respective feed conduit/tube 12, 22, with a respective first or second syringe pump device 11, 21 which contains a supply of the respective first or second composition comprising the respective first or second reactive substance, for controlled introduction into the system. Each respective syringe pump device 11, 21 comprises a respective piston 11P, 21P in a respective cylinder 11C, 21C, and is controlled by a respective suitable electronic or computer-based control device 11E, 21E, whereby each respective syringe pump device 11, 21 may be actuated and controlled to deliver its respective first or second composition into the system in a predetermined controlled manner—in particular in a predetermined carefully controlled amount or at a predetermined flow rate, e.g. a flow rate of around 0.2 to 0.3 ml/min for use with a 0.6 mm internal diameter nozzle device for the formation of relatively large first or second bodies (e.g. with diameters in the region of about 1-5 mm), or a flow rate of around 0.3 to 0.5 ml/min for use with a 0.6 mm internal diameter nozzle device for the formation of relatively small first or second bodies (e.g. with diameters in the region of about 0.05 or 0.1-1 mm). For instance, in the case of a pulsed flow obtained using a valve at the end of the or each nozzle 10N, 20N, a programmable computer-controlled relay or digital or mechanical timer (not shown) may be used to sequentially switch on and switch off the pump in a controlled manner, and so that each pump switches in parallel and the pump on-time determines the body volume that is delivered.

The apparatus 1 includes a high-voltage charge generator device 30, in order to deliver to nozzle 10N an EHT electric charge of one polarity (e.g. positive) and to nozzle 20N an EHT electric charge of the opposite polarity (e.g. negative). The high-voltage generator device 30, which is mains-powered via an appropriate rectifier device, supplies its respective EHT+ and EHT− charges to the respective nozzles 10N, 20N via respective connecting wires 36, 38, and respective intermediate electrically conductive linking tubes 14, 24 to which the nozzles 10N, 20N are connected via their respective feed tubes or pipes 10T, 20T. Thus, as each respective liquid composition passes from its respective syringe pump device 11, 21 to the respective nozzle 10N, 20N, it is charged to the relevant electric EHT + or − voltage potential as supplied by the charge generator device 30 to the respective nozzle 10N, 20N. EHT voltages of from about 2 or 3 kV up to about 10 or 15 kV may be commonly employed, especially for the delivery of charged relatively large first and/or second bodies (e.g. with diameters in the region of about 1-5 mm), whereas EHT voltages of about 5 or 6 kV up to about 20 or 30 kV may be more suitable for the delivery of charged relatively small first and/or second bodies (e.g. with diameters in the region of about 0.05 or 0.1-1 mm).

In order to optimise the electrical characteristics and safety/operability of the system, especially so as to avoid the propensity for short-circuits to occur between the ends/tips of the two nozzles 10N, 20N, the two nozzles 10N 20N are arranged—i.e. supported or clamped using appropriate support hardware—so that their respective free ends/tips are separated by a separation distance S in the approx. range of from about 5 to 30 mm apart, and at a subtended separation angle A in the approx. range of from about 45 to 60°.

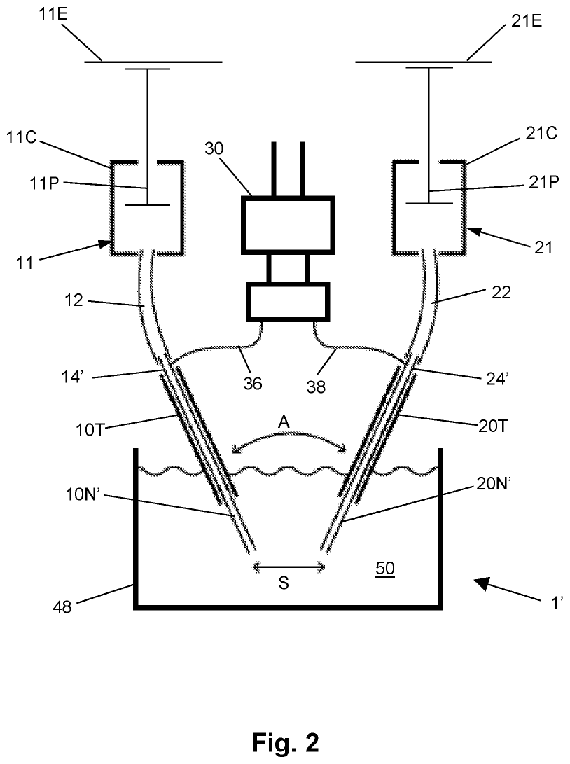

FIG. 2 shows schematically a modified basic set-up of reaction apparatus 1′ according to another embodiment of the invention [in which the same components as in the embodiment of FIG. 1 are designated by the same reference numeral but the modified components are designated using the same reference numeral but with a “'” suffix], in which a modified form of nozzle arrangement is employed. Here, instead of the respective connecting wires 36, 38 from the EHT generator 30 being connected to each respective nozzle 10N′, 20N′ via respective intermediate electrically conductive linking tubes 14′, 24′ (which are connected to the respective feed tubes or pipes 10T, 20T to which the nozzles 10N′, 20N′ are attached), in this modified embodiment each EHT+ and EHT− charge is delivered directly to each nozzle 10N′, 20N′ by virtue of each respective nozzle 10N′, 20N′ and respective intermediate electrically conductive linking tube 14′, 24′ being effectively united into a single integral conduit, tube or capillary , carried within the same respective feed tubes or pipes 10T, 20T.

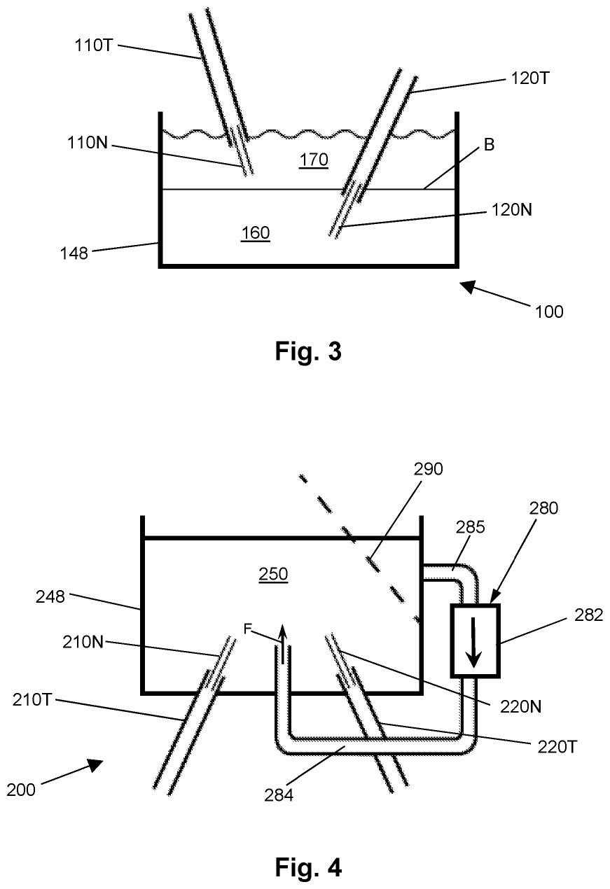

FIG. 3 shows schematically the lower region of another reaction apparatus 100 according to yet another embodiment of the invention [in which corresponding components as in the embodiment of FIG. 1 are designated by the same basic reference numeral but increased incrementally by 100], in which two discrete dielectric liquids 160, 170 are employed as a two-layer or two-phase dielectric medium. In this illustrative embodiment the upper phase 170 is deionised water and the lower phase 160 is an oily/hydrophobic heavier-than-water dielectric liquid such as triethyl citrate.

The upper nozzle 110N delivers into the upper aqueous phase 170 a charged first composition comprising the first reactive substance (from a corresponding first syringe pump device (not shown)) which may for example be an oily or hydrophobic or semiconductive such first composition, and the lower nozzle 120N delivers into the lower oily/hydrophobic phase 160 a charged second composition comprising the second reactive substance (from a corresponding second syringe pump device (not shown)) which may for example be an aqueous such second composition. Upon the delivery of each respective first and second composition into its respective dielectric liquid layer 160, 170, the resulting formed droplets, particles or capsules of the respective compositions thus undergo electrostatic attraction, thereby drawing them together towards the boundary interface B between the two dielectric liquids 160, 170 and thereby at least initially focussing the resulting reaction between the first and second substances in that boundary region B of the overall dielectric medium.

Of course, in alternative embodiments of a dual-dielectric-layer system such as that shown in FIG. 3, the physical nature of the two dielectric fluid layers 160, 170 could be reversed by appropriate selection of the densities of the two dielectric liquids used, so that e.g. the aqueous phase is the lower phase/layer 160 and the oily/hydrophobic phase is the upper phase/layer 170.

FIG. 4 shows schematically the lower region of another reaction apparatus 200 according to yet another embodiment of the invention [in which corresponding components as in the embodiments of FIGS. 1 & 3 are designated by the same basic reference numeral but again increased incrementally by 100], in which the nozzle arrangement is reversed in orientation and a pumping system 280 is employed to recirculate dielectric liquid 250 between lower and upper regions of the reaction container 248, as at F, in order to improve the stability of product bodies formed in the region between the two nozzles 210N, 220N.

In this embodiment the two nozzles 210N, 220N are located in a lower region of the reaction container 248, which here contains a single dielectric fluid layer 250, e.g. comprising a heavier-than-water oil phase (e.g. triethyl citrate). Each of the first and second compositions delivered into the system by the nozzles 210N, 220N (from corresponding first and second syringe pump devices (not shown)) may thus be an aqueous composition, so that the oppositely charged droplets, capsules or particles of those compositions tend to rise in the dielectric layer 250, as well as being electrostatically attracted to each other, and thus so that their electrostatically-induced mutual reaction is focussed (at least initially) in an upper region of the container 248.

In order to provide a stabilising upward-directed auxiliary, especially laminar, flow F of dielectric fluid 250 in the region of the dielectric medium between the nozzles 210N, 220N, i.e. a flow of dielectric fluid 250 in a direction away from the region therein at which the nozzles' outlets/mouths are closest together, a pumping system 280—comprising pump 282 and auxiliary recirculating pipework 284, 285—is employed to recirculate dielectric liquid 250 in that upward direction between the lower and upper regions of the reaction container 248.

In this manner, product bodies formed in the region between the two nozzles 210N, 220N are carried away from that region, especially so as to reduce the chances of unwanted clumping or coalescing thereof in the region between the nozzles 210N, 220N, and into the upper region(s) of the container 248 in a stable flowing manner, where they can more easily, stably and reliably interact to bring about the required electrostatically-induced reactions between the first and second substances in the respective first and second droplet/capsule/particle compositions.

As shown schematically FIG. 4, a perforated or dielectric-fluid-permeable and product-body-impermeable screen, filter or membrane 290 is provided adjacent the inlet to the upper recirculating pipe 285, in order to prevent product bodies in the upper region(s) of the dielectric medium 250 being recirculated through the pumping system 280.

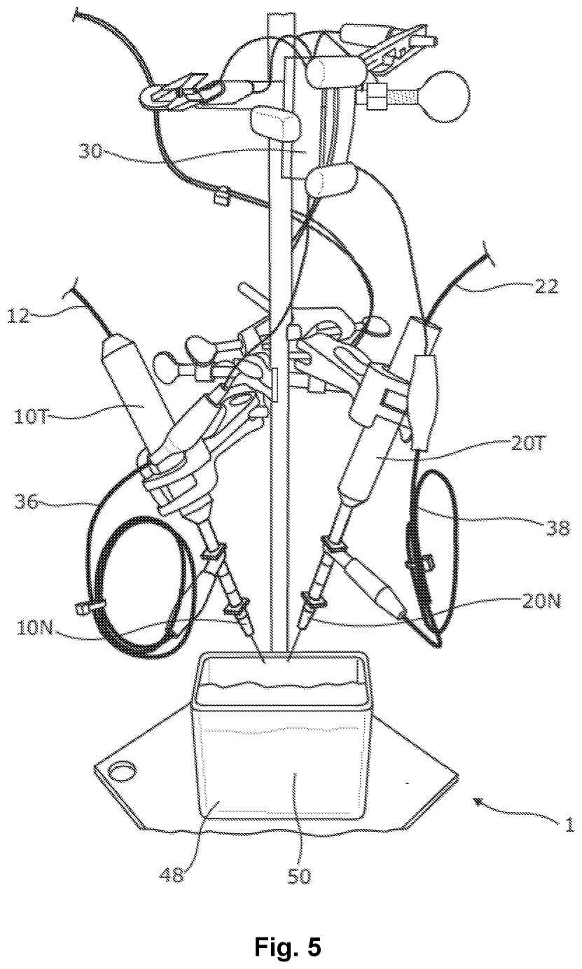

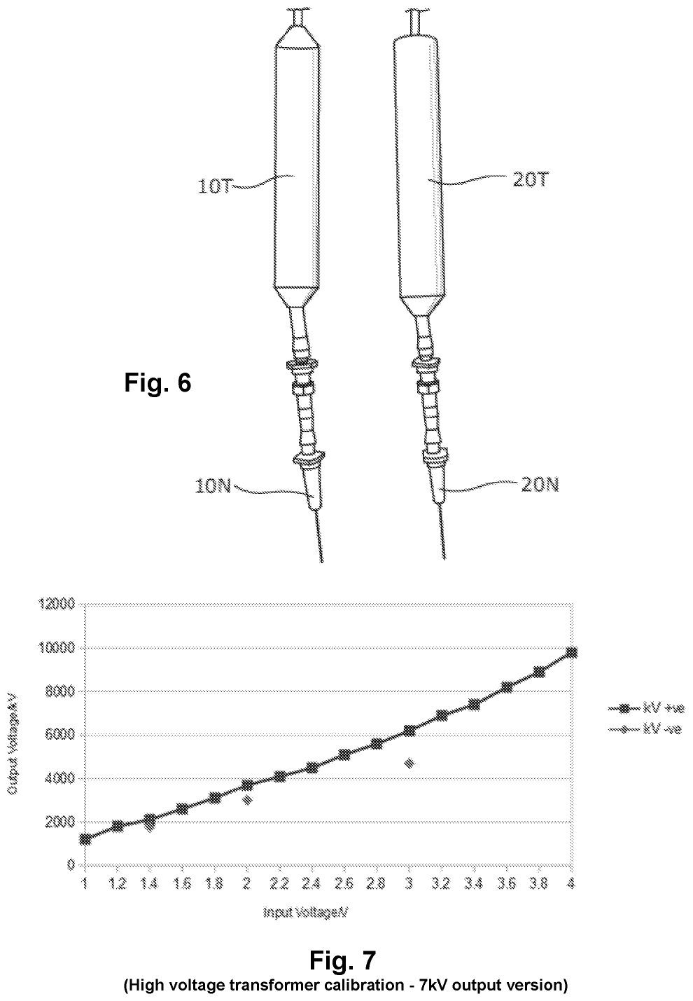

FIG. 5 is a photograph of an example prototype laboratory set-up of the apparatus shown schematically in FIG. 1, as used for conducting many of the working Example procedures as described in the working Examples hereinbelow, in which FIG. 5 the corresponding practical components as shown in the schematic embodiment of FIG. 1 are designated by the same reference numerals, and so will be self-explanatory as to their identity and function. FIG. 6 is a close-up photograph of the pair of composition delivery tubes 10T, 20T and nozzles 10N, 20N that are used to form the charged bodies of the first and second substance compositions in the apparatus of FIG. 5.

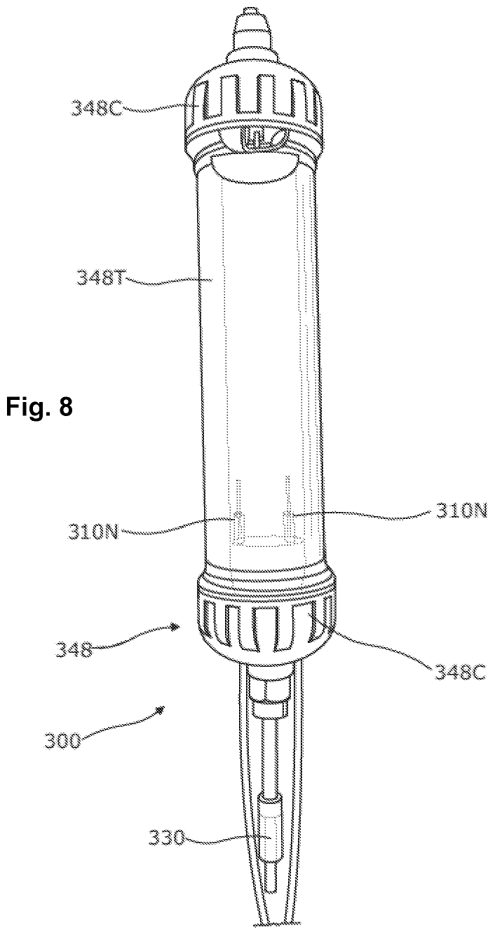

FIG. 8 is a side-on photograph of an alternative embodiment form of apparatus 300 within the scope of the invention, based on a different configuration of reaction vessel 348. Here, the reaction vessel 348 is in the form of a sealed reaction tube 348T with upper and lower end caps 348C, which contains a corresponding arrangement (in principle as in the preceding embodiments) of a pair of first and second substance composition delivery nozzles 310N, 320N, which are charged using a corresponding high-voltage charge generator device 330.

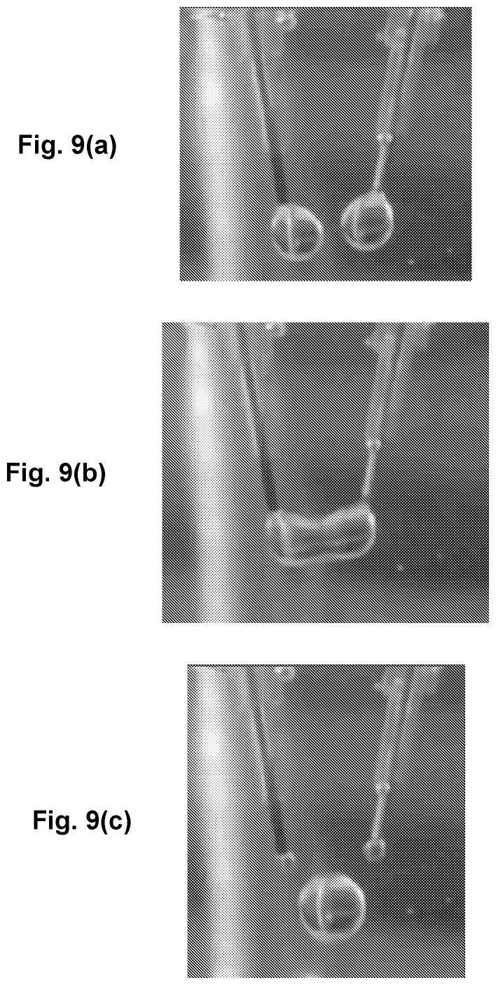

FIGS. 9(a), 9(b) & (9c) are photographs of representative examples of discrete oppositely-charged droplets of respective first and second compositions each containing a respective one of first and second reactive substances, which are formed using the prototype embodiment apparatus of FIG. 5 and are shown in various stages of their formation and deployment—namely in which the pair of droplets are: FIG. 9(a)—in the process of being formed; FIG. 9(b)—midway into being brought together and merging into a single product droplet; and FIG. 9(c)—having so merged and united into a single product droplet, undergoing chemical reaction between the first and second substances within the thus-produced product droplet.

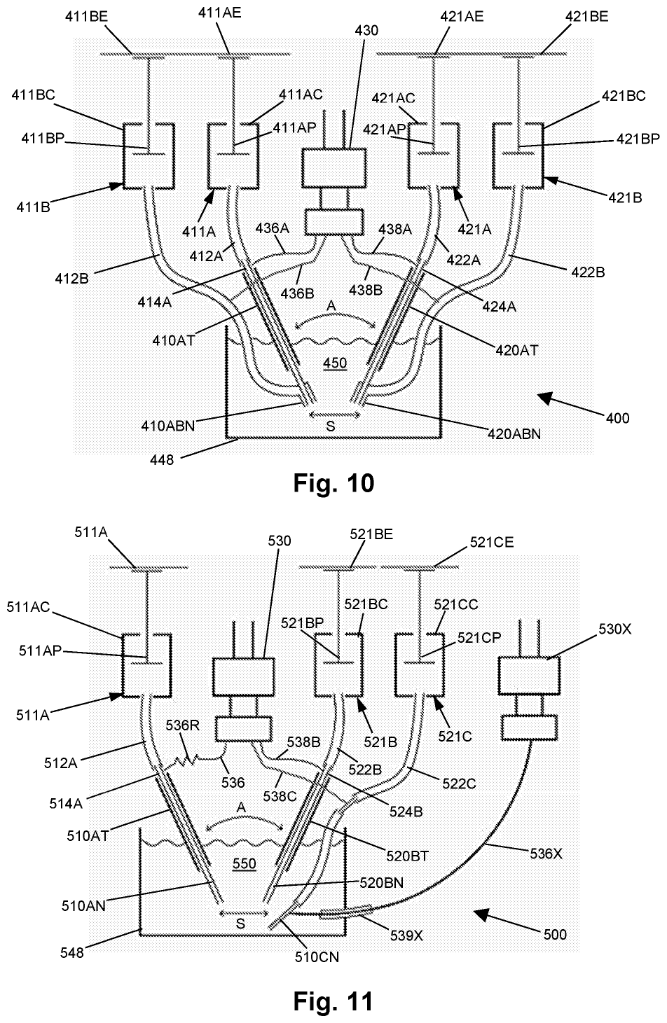

FIG. 10 shows schematically a more advanced form of reaction apparatus 400 according to yet another embodiment of the invention [in which corresponding components as in the embodiments of FIGS. 1 & 3 are designated by the same basic reference numeral but again increased incrementally by 100], in which a further modified form and arrangement of a pair of combined delivery nozzles 410ABN, 420ABN is employed, each of which produces concentric core-within-a-shell bodies of the first and second substance compositions.

A first combined nozzle 410ABN now comprises an elongated electrically conductive inner nozzle tube 414A delivering a first, “core” substance composition from a first syringe pump device 411A via feed conduit/tube 412A and associated feed tube or pipe 410AT. The first syringe pump device 411A comprises a first piston 411AP in a respective first cylinder 411AC and is controlled by a first electronic or computer-based control device 411AE. Likewise, an elongate, flexible but electrically conductive outer nozzle tube 412B delivers a second, “sheath/shell” substance composition from a second syringe pump device 411B, comprising a second piston 411BP in a respective second cylinder 411BC and is controlled by a second electronic or computer-based control device 411BE.

In a corresponding manner, a second combined nozzle 420ABN now comprises an elongated electrically conductive inner nozzle tube 424A delivering a first, “core” substance composition from a first syringe pump device 421A via feed conduit/tube 422A and associated feed tube or pipe 420AT. The first syringe pump device 421A comprises a first piston 421AP in a respective first cylinder 421AC and is controlled by a first electronic or computer-based control device 421AE. Likewise, an elongate, flexible but electrically conductive outer nozzle tube 422B delivers a second, “sheath/shell” substance composition from a second syringe pump device 421B, comprising a second piston 421BP in a respective second cylinder 421BC and is controlled by a second electronic or computer-based control device 421BE. The respective flowrates of the first and second substance compositions delivered to each core and each sheath/shell within each of the first and second combined nozzles 410ABN, 420ABN may be same or different, e.g. depending on the chemistry of the various components of the overall system and the respective electrostatic attractions required between the first and second reactive substance compositions delivered by each respective core-and-sheath/shell combination and as between the two core-and-sheath/shell combinations delivered by the two combined nozzles 410ABN, 420ABN.

The apparatus 400 includes a high-voltage charge generator device 430, which delivers to the first combined nozzle 410ABN, via connecting wires 436A, 436B, the required high voltage +ve and −ve charges to the respective inner nozzle tube 414A and outer nozzle tube 412B. Likewise it also delivers to the second combined nozzle 420ABN, via connecting wires 438A, 438B, the required high voltage +ve and −ve charges to the respective inner nozzle tube 424A and outer nozzle tube 422B. By arranging for the respective +ve and −ve charges applied to the respective (i) inner nozzle tube 414A and outer nozzle tube 412B, and (ii) inner nozzle tube 424A and outer nozzle tube 422B to be different from each other, a residual charge difference may be established between the respective core-and-sheath/shell combined bodies generated by each combined nozzle 410ABN, 420ABN, thereby enabling the resulting electrostatic attraction between those core-and-sheath/shell combined bodies from each nozzle to bring them together for the purpose of inducing further, or alternative, chemical reactions between their respective reactive substance constituents. (Of course in other specific embodiments, it may be possible for the polarities of the electric charges applied to each core and sheath/shell in each combined nozzle 410ABN, 420ABN to be reversed, so that each inner nozzle tube 414A, 424A is the −ve pole and each outer nozzle tube 412B, 422B is the +ve pole.)

Thus, in the illustrated FIG. 10 embodiment, electrostatically-induced reactions between the respective first and second substances in their respective compositions may take place not only between the core and sheath/shell bodies of each respective delivered combined droplet/capsule/particle from each of the respective combined nozzles 410ABN, 420ABN, but also as between those respective pairs of combined droplets/capsules/particles from the two combined nozzles 410ABN, 420ABN.

In a modification to this arrangement it may be possible for the combined droplets/capsules/particles from each respective one of the combined nozzles 410ABN, 420ABN to themselves contain different first and second substances from each other, whereby it may be possible to arrange for electrostatically-induced reaction between up to four such reactive substances, e.g. from two pairs of different first and second substances.

In a further alternative modification to this arrangement, one of the combined nozzles 410ABN or 420ABN could be replaced with a single nozzle—e.g. in principle corresponding to one of the nozzle devices 10N or 20N in FIG. 1 or 10N′ or 20N′ in FIG. 3—such as for introducing a single additional reactive substance (e.g. a cross-linking agent) into a system comprising just one combined core-within-a-sheath/shell species of droplet/capsule/particle supplied by the remaining one combined nozzle 410ABN or 420ABN.

FIG. 11 shows schematically another more advanced form of reaction apparatus 500 according to yet another embodiment of the invention [again, in which corresponding components as in the preceding embodiments are designated by the same basic reference numeral but again increased incrementally by 100], in which an additional third nozzle 510CN—in addition to first 510AN and second 520BN nozzles—is employed for forming and delivering into the reaction system one or more third bodies comprising a third substance and charged with an electric charge different in polarity from that of a residual charged product body resulting from the reaction between the first and second bodies delivered by the first and second nozzles 510AN, 520BN. Thus, in principle the embodiment apparatus of FIG. 11 may be thought of a modification of the embodiment apparatus 1′ of FIG. 3, in which the second charging and delivering nozzle 20N′ has been replaced with a combination of (i) a second charging and delivering nozzle 520BN, for delivering bodies of a second reactive substance composition (as before) for electrostatically-induced reaction with first bodies of a first reactive substance delivered by the first charging and delivering nozzle 510AN, and (ii) a third charging and delivering nozzle 510CN, for delivering bodies of a third reactive substance composition and charged with an electric charge different in polarity from that of a residual charged product body resulting from the reaction between the first and second bodies delivered by the first and second nozzles 510AN, 520BN.

The differential voltage is obtained by inserting a resistor 536R (e.g. a resistor of a suitable high resistance, or alternative a variable resistor) in the high voltage supply line 536 that supplies the first nozzle 510AN, which then acts as a voltage divider so as to reduce the voltage delivered to that first nozzle 510AN.

### EXAMPLES

In order to demonstrate the practical implementation and practising of embodiments of the present invention in its various aspects, various working Examples were carried out using the prototype apparatus of FIG. 5 to illustrate and demonstrate—by way of non-limiting representative examples only—some of the ways in which various first and second charged bodies which are suitable for containing and delivering first and second reactive substance compositions into the system for electrostatically-induced reaction together may be produced and controlled:

Equipment Specifications:
Syringe pump:
   Razel A-99 syringe pump, three off, acquired second hand; 0-99.9 setting equivalent to 0.324 ml/min on 69.9 setting (calibrated) and 0.461 ml/min on 99.9 setting (calibrated) for water at 20° C. for a 5 cm³ syringe.
Syringe:
   Becton Dickinson 5 ml polypropylene body with latex plunger, Luer-type syringe acquired from Fischer Scientific.
Metal Luer to barbed tube connector:
   from Cole Palmer.
Carbon fibre microbore tube nozzle/conductivity guide:
   Easy Composites pultruded carbon fibre tube in 0.3, 0.5, 0.7 and 1 mm internal diameter tubes.
Silicone tube, 2 mm inside diameter:
   from Advanced Fluid Solutions.
Polypropylene Luer to barbed pipe connector:
   Microflex, from Cole Palmer, 1/16 inch fitting.
Stainless nozzles:
   Logger Ltd sterile blunt no. 22 needles.
PTFE tube, ID 2 mm, OD 3 mm:
   from CRN Wholesale Ltd.
Regulated Power Supply:
   Rapid 85-1706, 0-30 V supply.
Voltage transformer:
   Made by: Jiangsu Tianwang Solar Technology Co., Ltd, No. 5, HuaXiang Road, Development Zone, Jingjiang City, Jiangsu Province, China. 0-3 V input gave 0-7 KV EHT+ and EHT− outputs, according to the supplier—but in practice for use in these experiments the unit required calibration, which calibration curve as measured was as shown in FIG. 7 of the accompanying drawings. Input voltages over 3 V increased output voltage proportionally, provided the output showed an electrical load. Calibrating this high voltage transformer was required for use in these experiments: this was conducted using an Amprobe 38X-A multimeter set to the DC voltage scale and connected to the source via a high voltage probe (Tenma 72-3040, 40 kV, 1000 MΩ input impedance with earth connection). The calibration curve was as shown in FIG. 7.
Nylon 66 electrode holder:
   Fabricated in-house from 16 mm bar stock, supplied by Nest Plastics Ltd.
Glass reactor tank:
   Slide staining jar from Wheaton (DWK Life Sciences).
The nozzle configuration was as shown in FIG. 6 of the accompanying drawings, and the overall reactor configuration was as shown in FIG. 5.

### Experiment 1—Reaction of Sodium alginate with Calcium Lactate to Form Microcapsules

Sodium alginate (food grade)—from Konrad Chemicals;

Calcium lactate (food grade)—from Sanus Vita Ltd;

Midel 7131—C5-10 esters of fatty acids with pentaerythritol, from M&I Materials Ltd, kinematic viscosity 29 mm2/sec at 37.8° C.

The reaction vessel was filled with Midel dielectric fluid. The nozzles were set to a separation distance of 13 mm and a separation angle of 24 degrees between the electrodes. The nozzles used were no. 22 needle, ID 0.41 mm, OD 0.71 mm. Syringe A (LHS +ve) was filled with a solution of calcium lactate (2 g in 120 g deionised water), and syringe B (RHS −ve) was filled with sodium alginate solution (1 g sodium alginate dissolved in 200 ml deionised water, settled and decanted). In all cases, unless otherwise defined, the polymer phase (alginate) was connected to the positively charged nozzle and the cross-linking aid (calcium lactate) was connected to the negatively charged nozzle. The tank was raised so that the stainless steel nozzles were 2 cm below the surface of the Midel fluid. Each syringe pump was set to 49.9, which equates to a flow rate of 0.229 ml/hr. Initially the regulated power supply input voltage was set to 1.8V and the output voltage to 3 kV, which produced large droplets of around 2-4 mm in diameter in a very stable manner with no satellites. Upon sequentially increasing the voltages, the regulated power supply input voltage was finally set to 4 V, 0.02 amps, which gave an output voltage at each nozzle of 9.8 kV, which generated a lot of turbulent mixing in the dielectric fluid producing smaller droplets of diameters <1 mm and producing many satellites.

For an output voltage at each nozzle of 3 kV it was observed that droplets slowly formed, dropped from each nozzle under the influence of electrostatic attraction and combined to mix forming a large drop which fell to the bottom of the tank. At the point of droplet combination calcium reacted with the alginate to form a shell and core structure with an alginate core and a calcium alginate shell. Shell structures under the conditions described here may have a weak mechanical strength, so can be easily squashed/burst by finger pressure.

At a nozzle voltage of 9.8 kV a stream of small droplets were emitted from each nozzle, causing a figure of eight turbulent recirculation around the nozzles resulting in small to very small particles being formed which fell to the bottom of the tank. On closer observation the liquid pool at the tank bottom was made up of many small capsules dispersed through the water phase. The water was unreacted calcium lactate solution.

The process was stable, continuing to form gel capsules within the size range 2-4 mm diameter with near zero satellite microcapsules being formed.

### Experiment 2

This was identical to Experiment 1, but with the addition of 1% Nonivamide (synthetic Capsaicin; sourced from Toronto Research Chemicals) added to the alginate phase and ground wet to form a slurry. It was ground using a Erko Dent Erkomix mixer, model 152.

Identical capsules were formed as those produced in Experiment 1.

### Experiment 3

Experiment 1 was repeated, but exchanging the Midel dielectric carrier oil with Sweet Almond oil with a trace of vitamin E, with kinematic viscosity 34.2 mm²/sec at 37.8° C. The source of the oil was Holland and Barrett, “Miaroma” brand. The flow rate to each nozzle was adjusted to 49.9 setting (i.e. 0.229 ml/min), and the regulated power supply set at 1.9 V, equating to 3.4 kV output, at 0.03 amps current.

Under these conditions 3 mm particles were formed from each nozzle, one by one in a very stable action.

### Experiment 4

This was identical to Experiment 3, except that the regulated power supply was increased to 2.2 V, equating to a transformer output voltage of 4.1 kV.

Under these conditions small capsules formed and recirculated in a figure-of-eight flow around the two nozzle electrodes. However, the particle-forming process was observed to be more turbulent, giving a variable particle size.

### Experiment 5

This was identical to Experiment 4, except that the nozzle separation distance was increased to 22 mm. The flow rate to each nozzle was also increased to 69.9 setting, i.e. 0.324 ml/min, and the regulated power supply output voltage was increased to 4 V, equating to a transformer output of 9.8 kV.

The highly turbulent flow resulted in microencapsulation of reactants similar to but of a smaller size distribution than those described in Experiment 4.

### Experiment 6

3 g of hemp oil containing 5% cannabidiol (CBD) (sourced from Jacob Hoop) was mixed with 5 g polypropylene glycol (sourced from R&R Laboratories Ltd), and 0.05 Polysorbate 80 (sourced from Acuchem Ltd). This mixture was thoroughly mixed to form an emulsion. The mixture was diluted 50:50 with deionised water and 2 ml of this solution was then mixed with 10 ml of the alginate solution described in Experiment 1 This mixture was charged by the +ve terminal of the transformer output.

The CBD/alginate was pumped from the positively charged nozzle and a calcium lactate solution was pumped through the negatively charged nozzle using the same calcium lactate composition as described in Experiment 1. The dielectric fluid was changed from Midel fluid to 100% caprylic acid (sourced from Primal Fuel) with a viscosity of 6.3 mm²/sec at 40° C.

The voltage output from the regulated power supply was increased stepwise from 1.8 up to 2.2, 4 and 7 V, equating to transformer outputs of 3.1, 4.1. 9.8 and 10 kV, respectively. The flow rate to each nozzle was set at 69.9 setting, equating to a flow rate of 0.324 ml/min.

It was observed that a transition occurred from the formation of single irregular (not spherical) particles at 1.8 V to an increasingly turbulent form of capsule formation at 7 V. The irregularity of particles generated at the lower nozzle voltage was a result of the high viscosity of the fluid which effected particle separation from the nozzle under the force generated by the electrostatic attraction between nozzles. Because of the narrow density difference between the aqueous phase and the caprylic acid phase, capsules formed by the combination of the materials delivered from nozzle 1 and nozzle 2 at the higher nozzle voltage recirculated around the nozzles without settling and passed through the electrostatic field several times, thereby building up a higher and higher concentration of aqueous-in-oil capsules. Above 2.2 V the capsules built up to form an emulsion in the oil phase. The emulsion was not stable, settling to form an aqueous layer below the oil layer. The aqueous layer contained within it a high concentration of microcapsules of sub-micron to 50-100 micron diameter sizes.

### Experiment 7

This was carried out using identical conditions and materials as described in Experiment 6, but using two sets of electrodes, each pair being spaced 20 mm apart, and powered off the same regulated power supply but using two separate high voltage transformers.

The nozzles proved not to interfere with each other's performance, increasing the rate of production of microcapsules in direct proportion to the flow rates through the nozzles.

### Experiment 8

3 g of hemp oil containing 5% cannabidiol (CBD) (sourced from Jacob Hoop) was mixed with 5 g polypropylene glycol (sourced from R&R Laboratories Ltd) and 0.05 Polysorbate 80 (sourced from Acuchem Ltd). This mixture was thoroughly mixed to form an emulsion. Each nozzle was changed to a carbon fibre microbore tube of 1.0 mm internal diameter. The highly viscous mixture was pumped from the positively charged nozzle at a rate setting of 99.99, equating to a flow rate of 0.462 ml/min. Calcium lactate solution as described in Experiment 1 was pumped from nozzle 2 at a rate setting of 99.99, equating to a flow rate of 0.462 ml/min.

Nozzle 1 formed a continuous thick thread which was electrically discharged by the impact therewith of droplets from nozzle 2. At the described flow rates the process was turbulent and unstable, but it was made more stable by reducing the flow rate through nozzle 2 delivering the calcium phase, namely to a flow setting of 69.9, equating to a flow rate of 0.324 ml/min. This produced a long continuous gel capsule with a 2-3 mm width. In this case the shell formed from the reaction of calcium lactate and sodium alginate forming insoluble calcium alginate, and the shell contained unreacted sodium alginate, hemp oil and polypropylene glycol.

### Experiment 9

This was identical to Experiment 2, but the Nonivamide was exchanged for 1% of a concentrated solution containing 2.3% Permethrin, 0.23% Tetramethryn and benzalkonium chloride (supplied by Rumenco under the “Net-tex” brand).

Identical capsules to those in Experiment 1 were formed.

### Experiment 10

This was identical to Experiment 2, but the Nonivamide was exchanged for 1% denatonium saccharide (supplied by Aquaricure ebay store), which was added to the alginate phase and ground wet to form a slurry. It was ground using an Erko Dent Erkomix mixer, model 152.

Identical capsules to those in Experiment 1 were formed.

### Experiment 11

This was identical to Experiment 2, but the Nonivamide was exchanged for 0.1% pure gibberelic acid (old stock supplied by ICI Biological Products), which was added to the alginate phase and ground wet to form a slurry. It was ground using an Erko Dent Erkomix mixer, model 152.

Identical capsules to those formed in Experiment 1 were formed.

### Experiment 12

The bottom entry reactor was used, as shown in FIG. 8 of the accompanying drawings, with triethyl citrate as the dielectric fluid (sourced from ebay seller, Labchem_de). Otherwise, the apparatus and process conditions and materials were identical to those of Experiment 1.

Triethyl citrate, being denser than water causes reaction products to float away from the reaction zone rather than settle to the bottom of the reactor in the case of the experiments previously described for Midel, vegetable and nut oils. Identical capsules to those in Experiment 1 were formed, with the exception that some capsules floated away from the reaction zone around the nozzles. However, on prolonged operation measured in minutes, sufficient water was absorbed by the triethyl citrate phase to make the dielectric fluid sufficiently semiconducting to allow discharge between nozzles. Alternative dielectric fluids which are denser than water but do not absorb water, such as mixtures of silicone oil and methylene chloride, may be expected to overcome or ameliorate this problem.

### Experiment 13

The bottom entry reactor was used, as shown in FIG. 8 of the accompanying drawings, with deionised water as the dielectric fluid The +ve pole was connected to the LHS nozzle and the −ve terminal was connected to the RHS nozzle. Peanut oil was used for droplet formation in both nozzles. The peanut oil incorporated 100 ppm Stadis 425 conductivity inducing agent (which was an old sample supplied by Dupont, now available from Innospec). The flow rate was set to 69.9 setting, which equates to 0.461 ml/min. The input voltage was set to 2.2 V, which equates to a 4.1 kV output.

Droplets were formed at the nozzle tips and were attracted to each other upon rising to the surface of the deionised water dielectric phase. In this Experiment the droplets did not react beyond being attracted to each other, at which point they discharged each other.

Re-running this Experiment 13 without any Stadis 425 caused arcing in the needle structure, due to a capacitive build-up of charge, and thus no attraction between droplets was observed.

This Experiment 13 was re-run with a water dielectric medium, using a water-in-oil emulsion for nozzle 1, made by homogenising peanut oil, a neutral aqueous solution of chitosan salicilate with a sucrose ester surfactant (Sucragel), and a water-in-oil emulsion for nozzle 2, made of peanut oil. The re-run experiment showed similar features to Experiment 12, wherein an initial stable capsule forming operation was followed by arcing across the nozzles due to absorption of electrolyte by the water phase. Electrolyte was released following the reaction of particles generated by nozzle 1 with particles generated by nozzle 2.

### Experiment 14

This was a re-run of Experiment 1, but substituting Chitosan in nozzle 1 and sodium tripolyphosphate in nozzle 2. Marine Chitosan of very high molecular weight was supplied by Cuantec. 36 g of Chitosan powder was dissolved in 30 ml of 1% acetic acid solution and heated in a microwave heater, and stirred until dissolved. To the resulting gel a further 120 ml of deionised water was added, and the pH was adjusted to 6.5. This solution was filtered through a 100 micron mesh filter. 2 g of sodium tripolyphosphate (from Mistral Chemicals) was dissolved in 50 ml deionised water. Flow rates were set to 69.9 setting, equating to 0.461 ml/min, and the voltage was set to 2.2 V, equating to 4.1 kV output. The nozzles were placed 13 mm apart.

It was observed that droplets formed at the nozzle tips, and were attracted to each other and reacted to form jelly-like chitosan capsules of 0.5-2 mm diameter, which collected at the bottom of the reactor. In this example the chitosan became cross-linked by contact with the tripolyphosphate forming a shell and core structure. The permeability of the cross-linked chitosan phase allowed the core to be completely cross-linked by inward diffusion of tripolyphosphate provided there was a stoichiometric excess and given sufficient time for the reaction to go to completion.

### Experiment 15

A two-phase dielectric fluid comprising Midel liquid and deionised water was placed carefully into the holding tank of the apparatus (as in the earlier Experiments), so that the water formed the lower layer/phase and the Midel liquid formed the upper layer/phase. The aqueous nozzle delivering into the oil phase was as described in Experiment 14 for Chitosan, which was connected to the +ve pole of the voltage supply. The oil nozzle delivering into the aqueous phase contained vanillin dissolved in soyabean oil, which was connected to the −ve pole of the voltage supply. The flow rate was adjusted to 0.461 ml/min, the voltage was adjusted to 4.1 kV output, and the nozzles were adjusted to 14 mm apart in the vertical dimension.

Droplets of Chitosan delivered into the Midel phase fell towards the oil water interface under gravity, accelerated by the charge on the oil phase droplets. Particles of the oil/vanillin phase were lighter than water, thereby rising to the oil-water interface, but there appeared to be no electrostatic attraction between the formed particles. Increasing the voltage to 10 KV did not induce electrostatic attraction, but caused arcing within the oil nozzle, indicating that the electrical conductivity of the oil phase was insufficient to induce electrohydrodynamic attraction and charge neutralisation.

The above Experiment 15 procedure was re-run homogenising 10 ppm Stadis 425 to the soyabean/vanillin phase, and thereafter inducing electrostatic attraction between drops generated in the oil and aqueous phases. This process was not stable due to the build-up of droplets at the oil water interface. However, it was expected that removal of some drops as they were formed would possibly increase the stability of the system.

### Experiment 16

This was a re-run of Experiment 2, but exchanging the 1% Nonivamide for 0.1% nano-copper (obtained from Promethean Particles). The nano-copper was hand-mixed into suspension to avoid particle-to-particle welding. The other apparatus and process conditions were identical to those in Experiment 2.

It was observed that dark-brown gel-like particles formed. Increasing flow rate and voltage as described in Experiment 6 produced results similar to Experiment 6, suggesting that the copper did not increase or decrease the electrical resistivity of the suspensions at a 0.1% concentration.

In a re-run of this Experiment 16 at higher nano-copper concentrations, at ˜5% the added copper made the phase sufficiently conductive for particle formation to occur, but the nano-copper could move into the oil phase (being highly oleophilic), which caused a breakdown of the process. This may potentially be able to be countered by coating the nano-copper with a chemical opposed oleophobing agent such as fluorosiloxane.

### Experiment 17

Experiment 6 was re-run in two further experiments, using CBD alone and carrageenan gum alone in place of the CBD.

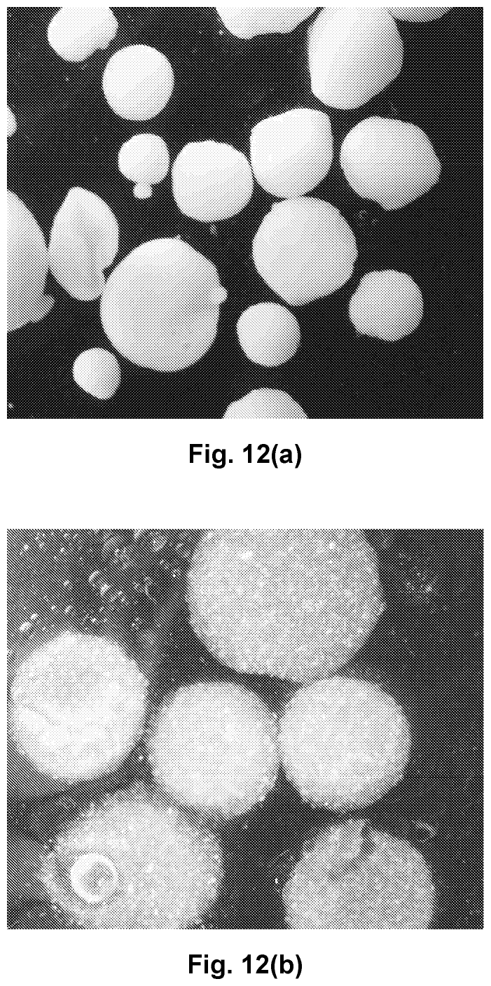

In each respective case, discrete cannabinoid or carrageenan approx. spherical microcapsules were able to be formed—both with sizes in the approx. range 3 mm-6 mm diameter. FIGS. 12(a) & 12(b) of the accompanying drawings are photographs of these cannabinoid and carrageenan product body species thus produced.

### Experiment 18

An experiment corresponding generally to Experiment 1 was carried out, but modified to illustrate the use of a gas generating agent (ammonium bicarbonate) in a system that forms shell-and-core type product bodies.

The dielectric fluid was silicone oil. Reactant A was a solution of sodium metaborate in water, adjusted to pH 1 with 5% acetic acid. Reactant B was a solution of polyvinyl alcohol and ammonium bicarbonate in water. Determining which phase made up the shell and which phase made up the core depended on which polarity nozzle reactant A and reactant B were delivered from.

For particles which had PVA/bicarbonate as the core materials, typically delivered from the +ve nozzle, a shell was formed by a cross-linking reaction between the PVA and borate, the borate/acid mixture being delivered from the −ve nozzle. Simultaneously acetic acid which was admixed with the borate reacted with bicarbonate which was admixed with the PVA, liberating carbon dioxide gas which caused capsules so formed to float away from the reaction zone. Alternatively, if the nozzle polarity was reversed, so that the borate/acetic acid made up the core (+ve electrode) and the PVA/bicarbonate the shell (−ve electrode), then substantially liquid-filled capsules were formed with a gas-filled and borate cross-linked PVA shell.

### Experiment 19

The following Example (corresponding generally to Experiment 1 but modified appropriately) demonstrates the use of a dielectric pseudo-fluid (i.e. a “liquid-on-powder” solid composition that behaves like a fluid) as the dielectric medium.

Silica Aerogel—from Aerogel UK Ltd, of bulk density 80 kg/m³;

Aqueous 25% shellac solution—SWANLAC ASL 10, from AF Sauter Ltd;

1% acetic acid solution—from Sigma Aldrich.

Silica aerogel was poured in to fill the reaction vessel. Fumed silica pours like a fluid. The nozzles were set to a separation distance of 13 mm and a separation angle of 24 degrees between the electrodes. The nozzles used were no. 22 needles, ID 0.41 mm, OD 0.71 mm. Syringe A (LHS +ve) was filled with a 1:4 diluted solution of SWANLAC 10 in deionised water, and syringe B (RHS −ve) was filled with 1% acetic acid solution. The polymer phase (shellac) was connected to the positively charged nozzle and the cross-linking aid (acid) was connected to the negatively charged nozzle. The tank was raised so that the stainless steel nozzles were 2 cm below the surface of the aerogel. Each syringe pump was set to 49.9, which equates to a flow rate of 0.229 ml/hr. The regulated power supply was set to 4 V, 0.02 amps, which equates to a voltage at each nozzle of 9.8 kV.

The presence of the opaque aerogel phase made direct observations impossible, but it was observed that rigid pellets of acid cross-linked SWANLAC accumulated at the bottom of the reactor within the aerogel phase. It was surmised that droplets formed, were electrostatically attracted and reacted in a manner to that observed using liquid dielectrics.

The process was stable, continuing to form gel capsules within the size range 2-4 mm diameter together with smaller satellite microcapsules of wide size distribution.

### Example 20

The following is another Example (corresponding generally to Experiment 1 but modified appropriately) demonstrating the use of a dielectric pseudo-fluid (i.e. a “liquid-on-powder” solid composition that behaves like a fluid) as the dielectric medium.

Sodium alginate (food grade)—from Konrad Chemicals;

Calcium lactate (food grade)—from Sanus Vita Ltd;

Silica aerogel was poured in to fill the reaction vessel. Fumed silica pours like a fluid. The nozzles were set to a separation distance of 13 mm and a separation angle of 24 degrees between the electrodes. The nozzles used were carbon fibre microbore tube of 1.0 mm internal diameter. Syringe A (LHS +ve) was filled with a solution of sodium alginate in deionised water (1 g sodium alginate dissolved in 200 ml deionised water, settled and decanted), and syringe B (RHS −ve) was filled with a solution of calcium lactate in deionised water (2 g in 120 g deionised water). The polymer phase (alginate) was connected to the positively charged nozzle and the cross-linking aid (calcium) was connected to the negatively charged nozzle. The tank was raised so that the stainless steel nozzles were 2 cm below the surface of the aerogel. Each syringe pump was set to 49.9, which equates to a flow rate of 0.229 ml/hr. The regulated power supply was set to 4 V, 0.02 amps, which equates to a voltage at each nozzle of 9.8 kV.

The presence of the opaque aerogel phase made direct observations impossible, but it was observed that rigid pellets of calcium cross-linked alginate accumulated at the bottom of the reactor. It was surmised that droplets formed, were electrostatically attracted and reacted in a manner to that observed using liquid dielectrics.

The process was stable, continuing to form gel capsules within the size range 3-5 mm diameter together with a small number of satellite microcapsules of wide size distribution.

Throughout the description and claims of this specification, the words “comprise” and “contain” and linguistic variations of those words, for example “comprising” and “comprises”, mean “including but not limited to”, and are not intended to (and do not) exclude other moieties, additives, components, elements, integers or steps.

Throughout the description and claims of this specification, the singular encompasses the plural unless expressly stated otherwise or the context otherwise requires. In particular, where the indefinite article is used, the specification is to be understood as contemplating plurality as well as singularity, unless expressly stated otherwise or the context requires otherwise.

Throughout the description and claims of this specification, features, components, elements, integers, characteristics, properties, compounds, chemical moieties or groups described in conjunction with a particular aspect, embodiment or example of the invention are to be understood to be applicable to any other aspect, embodiment or example described herein unless incompatible therewith or expressly stated otherwise.

1. A method for bringing together one or more first bodies comprising a first substance with one or more second bodies comprising a second substance for the purpose of the first and second substances bodies, or the first and second substances contained therein, reacting together, the method comprising:
forming the said one or more first bodies with an electric charge of a first polarity applied thereto, and forming the said one or more second bodies with an electric charge of a second polarity applied thereto, the first polarity being opposite to the second polarity, whereby the oppositely polarised electric charges on the first and second bodies causes or promotes electrostatic attraction between one or more respective pairs of the one or more first bodies and the one or more second bodies,
wherein the one or more charged first bodies and the one or more oppositely charged second bodies are each formed in a dielectric medium comprising one or more dielectric materials,
and the one or more charged first bodies and the one or more oppositely charged second bodies each have a size or width of at least about 0.01 mm or greater, especially of at least about 0.02 or 0.03 or 0.04 or 0.05 mm or greater.

2. A method according to claim 1, wherein the reacting together of the first and second bodies comprises:
either (i) a joining or merging or combining together of the first and the second bodies so as to form one or more product bodies, wherein the one or more product bodies comprise both the first and the second substances, which first and second substances are thereby able to interact and/or react together within the one or more product bodies, whereby the one or more product bodies resulting from the joining or merging or combining together of the first and the second bodies comprise a product of a chemical interaction or reaction between the first and the second substances originally present in the first and second bodies, respectively;
or (ii) a joining or merging or combining together of the first and the second bodies so as to form one or more product bodies, wherein the one or more product bodies comprise discrete portions or components (especially of the nature of a shell-and-core or encapsulated droplet or particle or body structure), each of which portions or components contains a respective one of the first and second substances, but which first and second substances remain separate from each other within the one or more product bodies, whereby the one or more product bodies resulting from the joining or merging or combining together of the first and the second bodies comprise a product of a physical interaction or reaction between the first and the second bodies, rather than a chemical one between the first and second substances contained therein.

3. A method according to claim 1, wherein the dielectric medium is in the form of a dielectric fluid or a dielectric pseudo-fluid (i.e. a dielectric composition that comprises one or more solid substances but which behaves like a fluid);
optionally wherein the dielectric fluid or pseudo-fluid has a specific resistance in the range of from 10⁻⁸ to 10⁻¹⁵ Ωm, and/or and an electric breakdown voltage/potential in the range of from 150 V/mm to 90 kV/mm;
and further optionally wherein the dielectric medium comprises one or more dielectric liquids, and the or each dielectric liquid has a kinematic viscosity in the range of from 1 cSt to 30 cSt.

4-5. (canceled)

6. A method according to claim 3, wherein either:
(i) the, or at least one of the, dielectric fluid(s) or pseudo-fluid(s) has a density which is lower than that of water; or
(ii) the, or at least one of the, dielectric fluid(s) or pseudo-fluid(s) has a density which is higher than that of water.

7. A method according to claim 3, wherein either:
(i) the, or at least one of the, dielectric fluid(s) or pseudo-fluid(s) is hydrophobic or oliophilic; or
(ii) the, or at least one of the, dielectric fluid(s) or pseudo-fluid(s) is hydrophilic or oliophobic.

8. A method according to claim 1, wherein:
either (i) the dielectric medium comprises one or more dielectric liquids selected from one or more of any of the following:
silicones or silicone oils;
hydrocarbons;
halogenated hydrocarbons;
fluorinated or perfluorinated organic compounds;
perether fluorocarbons;
esterified natural or vegetable oils;
reactive methyl hydrogen siloxate;
mineral oils;
castor oil;
vegetable, nut or seed oils;
polyphosphazines;
medium-chain fatty acids;
carboxylic and other acid esters;
admixtures of oils with other organic solvents;
super-oleophobic materials being fluorinated surfactants;
deionised water;
or (ii) the dielectric medium comprises one or more dielectric pseudo-fluids (i.e. a dielectric composition that comprises one or more solid substances but which behaves like a fluid), wherein the or each dielectric pseudo-fluids comprises a dielectric composition which comprises one or more particulate or powdered solid substances comprising or carrying one or more liquid or solid dielectric materials.

9. (canceled)

10. A method according to claim 1, wherein either:
(i) the dielectric medium comprises substantially just a single dielectric fluid or pseudo-fluid, whereby the charged bodies of the first substance are formed in the same phase, layer or volume of the dielectric medium as are formed the charged bodies of the second substance; or
(ii) the dielectric medium comprises a plurality of different dielectric fluids or pseudo-fluids, wherein each dielectric fluid or pseudo-fluid forms, or is present in the container of the apparatus as, its own discrete phase or layer or volume which is separate or discrete from the phase, layer or volume forming the other, or at least one or more of the other, dielectric fluids or pseudo-fluids, whereby the charged bodies of the first substance are formed in a different phase, layer or volume of the dielectric medium from the phase, layer or volume in which are formed the charged bodies of the second substance.

11. A method according to claim 1, wherein each of the first bodies formed comprising the first substance and each of the second bodies formed comprising the second substance are each independently formed so as to take the form of a droplet, globule, capsule, particle, chain, thread or fibre.

12. A method according to claim 1, wherein at least one of the first or second body species is formed as a core body and at least one of the other first or second body species is formed as a hollow or shell body which surrounds or encapsulates or enshrouds or encases the core body.

13. A method according to claim 1, wherein the first charged bodies and/or the second charged bodies each have a size or width (i.e. diameter or transverse width) in a range of from 0.01 or 0.02 or 0.03 or 0.04 or 0.05 or 0.06 or 0.07 or 0.08 or 0.09 or 0.1 or 0.2 or 0.3 or 0.4 or 0.5 mm up to 2 or 3 or 5 or 6 or 7 or 8 or 9 or 10 or 15 or 20 mm.

14. A method according to claim 1, wherein the one or more first bodies comprise a first liquid composition including the said first substance and optionally one or more auxiliary components, and the one or more second bodies comprise a second liquid composition including the said second substance and optionally one or more auxiliary components;
optionally wherein:
(i) the first composition is selected from a substantially aqueous or hydrophilic composition, or a substantially oily or hydrophobic composition, or an electrically conductive composition, or a substantially electrically non-conductive composition, or a substantially electrically semiconductive composition; and independently thereof
(ii) the second composition is selected from a substantially aqueous or hydrophilic composition, or a substantially oily or hydrophobic composition, or an electrically conductive composition, or a substantially electrically non-conductive composition, or a substantially electrically semiconductive composition;
and further optionally wherein at least one of, optionally either or each or both of, the first composition and/or the second composition includes one or more auxiliary component substances selected from:
(iii) chemically inert auxiliary substances for altering or adjusting one or more physical properties of the respective composition in which it is incorporated, selected from: an inert powder or particulate material to adjust viscosity, or a buffering agent to adjust or maintain a desired pH, or magnetic particles to enable product bodies to be manipulated using a magnetic field in order to move them into a location in the dielectric medium at which they may be more readily collected and/or recovered, or electrically conductive particles to adjust the electrical conductivity characteristics of the composition into which it is incorporated; or
(iv) chemically reactive substances which are involved in a chemical reaction between the first and second reactive substances, selected from: a co-reactant, initiator or catalyst or other promoter of the main chemical reaction between the first and second reactive substances.

15-16. (canceled)

17. A method according to claim 1, wherein the reaction between the first bodies and the second bodies, or of the first and second substances contained therein, takes place, after they have been brought together by the electrostatic attraction between them, under conditions in which free oxygen is substantially absent from/in the said dielectric medium in which the charged first and second bodies are formed.

18. A method according to claim 1, wherein product bodies resulting from the bringing together of, and reaction between, the first and second bodies themselves have or carry a residual electric charge.

19. Apparatus for bringing together one or more first bodies comprising a first substance with one or more second bodies comprising a second substance for the purpose of the first and second bodies, or the first and second substances contained therein, reacting together, the apparatus comprising:
a container for containing, or containing, a dielectric medium comprising one or more dielectric materials;
first means for forming the said one or more first bodies comprising the first substance and applying thereto an electric charge of a first polarity, and second means for forming the said one or more second bodies comprising the second substance and applying thereto an electric charge of a second polarity, the first polarity being opposite to the second polarity;
wherein the first and second forming means are each arranged for forming the respective said one or more charged first bodies and one or more charged second bodies such that the one or more charged first bodies and the one or more oppositely charged second bodies each have a size or width of at least about 0.01 mm or greater, especially of at least about 0.02 or 0.03 or 0.04 or 0.05 mm or greater, and such that each thereof is located in the said dielectric medium once contained, or that is contained, in the container;
whereby, once formed in the said dielectric medium, the oppositely polarised electric charges on the said first and second bodies causes or promotes electrostatic attraction between one or more respective pairs of the one or more first bodies and the one or more second bodies;
optionally wherein the container which forms the reaction vessel includes separation or collection means for enabling product bodies resulting from the bringing together of, and reaction between, the first and second bodies to be collected and separated out from the dielectric medium, ready for subsequent removal or extraction from the container or reaction vessel.

20. (canceled)

21. Apparatus according to claim 19, wherein:
(i) the first means for forming the said one or more first bodies comprising the first substance and applying thereto an electric charge of a first polarity comprise a first nozzle device and a first charging means, optionally wherein either (ia) the first nozzle device and first charging means are provided in combination with one another in a single substantially unified first delivery means which serves both purposes, or (ib) the first nozzle device and the first charging means are provided as discrete components of a two-stage or two-element first delivery means of which each discrete element effects a respective one of the first body-forming and the first body-charging functions; and independently thereof
(ii) the second means for forming the said one or more second bodies comprising the second substance and applying thereto an electric charge of a second polarity comprise a second nozzle device and a second charging means, optionally wherein either (iia) the second nozzle device and second charging means are provided in combination with one another in a single substantially unified second delivery means which serves both purposes, or (iib) the second nozzle device and the second charging means are provided as discrete components of a two-stage or two-element second delivery means of which each discrete element effects a respective one of the second body-forming and the second body-charging functions;
optionally wherein either or each of the first and second nozzle devices, together with their associated or integrated respective first and second charging means, forms and ejects a respective one or more of, optionally a group or cluster or cloud or series or sequence or chain of a plurality of, the respective oppositely charged first and second bodies by virtue of:
(iii) an electrohydrodynamic comminution process; or
(iv) a simple droplet- or particle-formation and release (or emission) process or step.

22. (canceled)

23. Apparatus according to claim 21, wherein either or each of the first and/or second nozzle device(s) comprises a respective outlet or mouth connected to a respective feed tube, conduit, pipe, capillary or channel which is in fluid communication with a respective reservoir containing a supply of the respective first or second composition comprising or including the respective first or second reactive substance;
optionally wherein one or more of the following (i), (ii) and (iii) is present or satisfied:
(i) the outlet or mouth of each respective nozzle device is in cross-section substantially circular, elliptical, rectangular, square, polygonal, or slot- or slit-shaped;
(ii) the outlet or mouth of each respective nozzle device is fitted or provided or formed with an exit flow control feature or attachment or a valve;
(iii) the outlet or mouth of each respective nozzle device has an internal width in the range of from about 0.01 or 0.05 or 0.1 mm up to about 2 or 3 or 5 or 6 or 7 or 8 or 9 or 10 mm.

24. (canceled)

25. Apparatus according to claim 23, wherein the or each respective reservoir is provided with, or includes, feed or injection or pump means for delivering or forcing the respective first or second composition to the respective nozzle device's outlet/mouth at a predetermined flow rate or speed or in a predetermined dose amount or volume per given delivery operation, step or stage;
optionally wherein each respective one of the first and second compositions is delivered to its respective nozzle device's outlet/mouth at a flow rate in a range of from about 0.1 or 0.15 or 0.2 or 0.25 or 0.3 or 0.4 or 0.5 or 0.6 or 0.7 or 0.8 ml/min up to about 5 or 6 or 7 or 8 or 9 or 10 ml/min, optionally from about 0.1 or 0.15 or 0.2 or 0.25 or 0.3 or 0.4 or 0.5 or 0.6 ml/min up to about 1 or 1.5 or 2 ml/min.

26. (canceled)

27. Apparatus according to any one of claim 21, wherein either or each of the first and/or second charging means, which apply the said electric charges of opposite polarities to the formed first and second bodies, comprises a respective electric charge source or charge delivery device, and each said electric charge source or charge delivery device supplies an electric voltage of from about 1 kV up to about 20 or 30 kV.

28. Apparatus according to claim 23, wherein either:
(i) the dielectric medium comprises just a single dielectric fluid or pseudo-fluid, and both of the respective outlets or mouths of the respective nozzle devices that deliver or eject their respective first and second body(ies) are submerged in the said dielectric fluid or pseudo-fluid forming the dielectric medium, whereby the charged first bodies are formed in the same phase, layer or volume of the dielectric medium as are formed the charged second bodies; or
(ii) the dielectric medium comprises a plurality of different dielectric fluids or pseudo-fluids which form respective distinct separate phases or layers in the overall dielectric medium, and each of the respective outlets or mouths of the respective nozzle devices that deliver or eject their respective first and second body(ies) are submerged in either (iia) the same discrete phase or layer of a given one of the dielectric fluids or pseudo-fluids in the overall dielectric medium, or (iib) in respective different ones of the discrete phases or layers of the dielectric fluids or pseudo-fluids in the overall dielectric medium, whereby the charged first bodies are formed in a different discrete phase or layer of the dielectric medium from the discrete phase or layer thereof in which are formed the charged second bodies.

29. Apparatus according to claim 23, wherein the first and second nozzle devices are mounted or supported in the container containing the dielectric medium so as to be separated from each other and in an orientation and relative positioning or configuration such that:
(i) the first and second nozzle devices are mounted or supported relative to each with a distance separation at their respective free ends/tips in the range of from about 3 or 4 or 5 or 6 or 7 or 8 mm up to about 10 or 12 or 15 or 20 or 25 or 30 mm; and/or
(ii) the first and second nozzle devices are mounted or supported relative to each with an angle separation (i.e. the angle subtended by the longitudinal axes of the two nozzle devices, relative to each other) in the range of from about 45° up to about 120°, optionally from about 50 or 60° up to about 90 or 100 or 110°;
optionally wherein the apparatus further includes means for providing an auxiliary flow of dielectric fluid or pseudo-fluid within the overall dielectric medium away from the region at which the nozzles' outlets/mouths are closest together.

30. (canceled)

31. Apparatus according to claim 23, wherein the first and second nozzle devices are constructed and configured or arranged so as to be concentric or co-axial with respect to one another.

32. Apparatus according to claim 23, wherein one or more third or additional nozzles and associated or integral charging means are provided, for forming and delivering one or more third or additional charged bodies comprising a third substance into the dielectric medium, for reaction with either of, or both of, the first and/or second charged bodies.

33. A method for carrying out a chemical and/or physical reaction between one or more first bodies and one or more second bodies, or a first substance and a second substance contained respectively in the first and second bodies, the method comprising:
forming the one or more first bodies comprising the first substance and applying thereto an electric charge of a first polarity, and forming the one or more second bodies comprising the second substance and applying thereto an electric charge of a second polarity, the first polarity being opposite to the second polarity,
wherein the one or more charged first bodies and the one or more oppositely charged second bodies are each formed in a dielectric medium comprising one or more dielectric material, and the one or more charged first bodies and the one or more oppositely charged second bodies each have a size or width of at least about 0.01 mm or greater, especially of at least about 0.02 or 0.03 or 0.04 or 0.05 mm or greater;
allowing one or more respective pairs of the one or more charged first bodies and the one or more oppositely charged second bodies to move towards and come together with one another by virtue of electrostatic attraction therebetween caused or promoted by the oppositely polarised electric charges on the first and second bodies;
whereby the coming together of the one or more respective pairs of the one or more charged first bodies and the one or more oppositely charged second bodies enables the first and second bodies, or the first and second substances contained therein, to chemically and/or physically react together.
